(19) United States
(12) Patent Application Publication
(10) Pub. No.: US 2009/0317741 A1
(43) Pub. Date: Dec. 24, 2009

(54) COMPOUND, ACID GENERATOR, RESIST COMPOSITION AND METHOD OF FORMING RESIST PATTERN

(75) Inventors: Takeshi Iwai, Kawasaki-shi (JP); Makiko Irie, Kawasaki-shi (JP)

Correspondence Address: KNOBBE MARTENS OLSON & BEAR LLP, 2040 MAIN STREET, FOURTEENTH FLOOR, IRVINE, CA, 92614, US

(73) Assignee: Tokyo Ohka Kogyo Co., Ltd., Kawasaki-shi (JP)

(21) Appl. No.: 12/373,287

(22) PCT Filed: Jul. 3, 2007
(86) PCT No.: PCT/JP2007/063293

§ 371 Date: Jan. 9, 2009

(30) Foreign Application Priority Data
Jul. 14, 2006 (JP) 2006-194414
Nov. 10, 2006 (JP) 2006-305684

(51) Int. Cl. G03F 7/20; C07D 333/52; G03F 7/004

(52) U.S. Cl. 430/270.1; 549/43; 430/325

(57) ABSTRACT

There is provided a resist composition which includes a base component (A) which exhibits changed solubility in an alkali developing solution under action of acid, and an acid generator component (B) which generates an acid upon exposure, wherein the acid generator component (B) comprises an acid generator composed of a compound represented by the general formula (b1-2) shown below:

(wherein, R⁴¹, R⁴², and R⁴³ each independently represents an alkyl group, an acetyl group, an alkoxy group, a carboxy group, or a hydroxyalkyl group; n₁ represents an integer of 0 to 3; n₂ and n₃ each independently represents an integer of 0 to 3; not all of n₁, n₂, and n₃ are simultaneously 0; and X⁻ represents an anion).

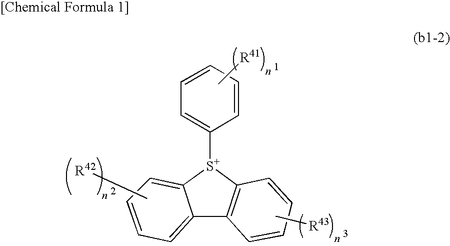

### TECHNICAL FIELD

The present invention relates to a compound suitable as an acid generator for a resist composition, an acid generator composed of the compound, a resist composition containing the acid generator, and a method of forming a resist pattern using the resist composition.

The application claims priority from Japanese Patent Application No. 2006-194414 filed on Jul. 14, 2006, and Japanese Patent Application No. 2006-305684 filed on Nov. 10, 2006, the disclosure of which is incorporated by reference herein.

### BACKGROUND ART

Lithography techniques include processes in which, for example, a resist film formed from a resist material is formed on top of a substrate, the resist film is selectively exposed with irradiation such as light, an electron beam or the like through a mask in which a predetermined pattern has been formed, and then a developing treatment is conducted, thereby forming a resist pattern of the prescribed shape in the resist film. Resist materials in which the exposed portions change to become soluble in a developing solution are termed positive materials, whereas resist materials in which the exposed portions change to become insoluble in the developing solution are termed negative materials.

In recent years, in the production of semiconductor elements and liquid crystal display elements, advances in lithography techniques have led to rapid progress in the field of miniaturization.

Typically, these miniaturization techniques involve shortening the wavelength of the exposure light source. Conventionally, ultraviolet radiation typified by g-line and i-line radiation has been used; however, nowadays, KrF excimer lasers and ArF excimer lasers are starting to be introduced in mass production of semiconductor elements. Furthermore, research is also being conducted into lithography techniques that use F₂ excimer lasers, electron beams (EB), extreme ultraviolet radiation (EUV) and X-rays.

Resist materials are required to have lithography properties such as high sensitivity to the aforementioned light source and enough resolution to reproduce patterns with very fine dimensions. As resist materials which fulfill the aforementioned requirements, there is used a chemically-amplified resist containing a base resin that displays changed solubility in an alkali developing solution under the action of acid, as well as an acid generator that generates an acid upon exposure. For example, a chemically-amplified positive resist includes, as a base resin, a resin which exhibits increased solubility in an alkali developing solution under the action of acid, and an acid generator. When an acid is generated from the acid generator upon exposure in the formation of a resist pattern, the exposed portions are converted to an alkali-soluble state.

Until recently, polyhydroxystyrene (PHS) or derivative resins (PHS-based resins) in which the hydroxyl groups have been protected with acid dissociable, dissolution inhibiting groups, which exhibit a high degree of transparency relative to KrF excimer laser (248 nm), have been used as the base resin of chemically-amplified resists. However, because PHS-based resins contain aromatic rings such as benzene rings, their transparency is inadequate for light with a wavelength shorter than 248 nm, such as light with a wavelength of 193 nm. Accordingly, chemically-amplified resists that use a PHS-based resin as the base resin have a disadvantage in that they have low resolution in processes that use, for example, light of 193 nm.

As a result, resins (acrylic resins) that contain structural units derived from (meth)acrylate esters within the main chain are now widely used as base resins for resists that use ArF excimer laser lithography, as they exhibit excellent transparency in the vicinity of 193 nm. In the case of a positive resist, as the base resin, those which have a structural unit derived from (meth)acrylate ester including an aliphatic polycyclic group-containing, tertiary alkyl ester-type acid dissociable, dissolution inhibiting group, such as a structural unit derived from 2-alkyl-2-adamantyl (meth)acrylate, are mainly used (for example, see Patent Document 1).

Here, the term “(meth)acrylate ester” is a generic term that includes either or both of the acrylate ester having a hydrogen atom bonded with the α-position and the methacrylate ester having a methyl group bonded with the α-position. The term “(meth)acrylate” is a generic term that includes either or both of the acrylate having a hydrogen atom bonded with the α-position and the methacrylate having a methyl group bonded with the α-position. The term “(meth)acrylic acid” is a generic term that includes either or both of the acrylic acid having a hydrogen atom bonded with the α-position and the methacrylic acid having a methyl group bonded with the α-position.

On the other hand, as acid generators used in a chemically-amplified resist, various types have been proposed including, for example, onium salt-based acid generators such as iodonium salts and sulfonium salts; oxime sulfonate-based acid generators; diazomethane-based acid generators; nitrobenzylsulfonate-based acid generators; iminosulfonate-based acid generators; and disulfone-based acid generators. Currently, as acid generators, those which include a triphenylsulfonium skeleton, dinaphthyl monophenylsulfonium skeleton, or the like are used (for example, see Patent Document 2).

[Patent Document 1] Japanese Unexamined Patent Application, First Publication No. 2003-241385.

[Patent Document 2] Japanese Unexamined Patent Application, First Publication No. 2005-100203.

### DISCLOSURE OF THE INVENTION

### Problems to be Solved by the Invention

In recent years, as requirements for high resolution increase with progress in the miniaturization of resist patterns, improvement in various lithography properties has been demanded.

As an example of such lithography properties, line width roughness (hereafter, frequently abbreviated as “LWR”) can be mentioned. LWR is a phenomenon in which the line width of a line pattern becomes uneven (non-uniform) when a resist pattern is formed using a resist composition, and improvement in the level of LWR becomes an important issue as pattern miniaturization progresses.

Further, as the cation for onium salt-based acid generators, a highly-hydrophobic cation, such as triphenylsulfonium and dinaphthyl monophenylsulfonium, is generally used. However, onium salt-based acid generators having such a cation has a problem in that the solubility thereof is low in an organic solvent (resist solvent) used for dissolving various components of a resist. Such low solubility in a resist solvent lowers the post exposure stability of the latent image formed by the pattern-wise exposure of the resist, thereby causing deterioration of the resist pattern shape.

The present invention takes the above circumstances into consideration, with an object of providing a novel compound suitable as an acid generator for a resist composition, an acid generator composed of the compound, a resist composition which includes the acid generator, and a method of forming a resist pattern using the resist composition.

### Means for Solving the Problems

The inventors of the present invention suggest the following in order to solve the above problem.

That is, the first aspect of the present invention is a compound represented by the general formula (b1-2) shown below.

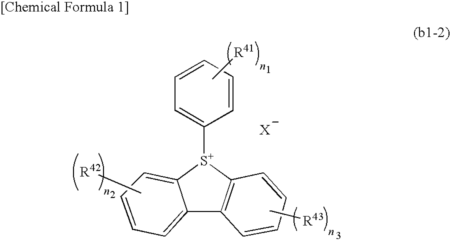

(In the formula, R⁴¹, R⁴², and R⁴³ each independently represents an alkyl group, an acetyl group, an alkoxy group, a carboxyl group, or a hydroxyalkyl group; n₁ represents an integer of 0 to 3; n₂ and n₃ each independently represents an integer of 0 to 3, where not all of n₁, n₂, and n₃ are simultaneously 0; and X⁻ represents an anion.)

The second aspect of the present invention is an acid generator composed of the compound represented by the above general formula (b1-2).

The third aspect of the present invention is a resist composition including a base component (A) which exhibits changed solubility in an alkali developing solution under action of acid and an acid generator component (B) which generates acid upon exposure, wherein

the acid generator component (B) includes the acid generator (B1) composed of the compound represented by the above general formula (b1-2).

The fourth aspect of the present invention is a method of forming a resist pattern, including: forming a resist film on a substrate using a resist composition of the third aspect of the present invention; exposing the resist film; and alkali-developing the resist film to form a resist pattern.

Here, the term “structural unit” means a monomer unit that contributes to the formation of a resin component (polymer).

The term “exposure” is used as a general concept involving irradiation with any form of radiation.

The term “alkyl group” is a concept containing a linear, branched and cyclic monovalent saturated hydrocarbon group, unless another definition is particularly provided.

The term “lower alkyl group” means an alkyl group of 1 to 5 carbon atoms. The term “lower alkyl group” in the term “halogenated lower alkyl group” means the same as “lower alkyl group” described above.

### EFFECTS OF THE INVENTION

According to the present invention, there are provided a novel compound suitable as an acid generator for a resist composition, an acid generator composed of the compound, a resist composition including the acid generator, and a method of forming a resist pattern using the resist composition.

### BEST MODE FOR CARRYING OUT THE INVENTION

### <<Compound>>

The compound according to the first aspect of the present invention is represented by the aforementioned general formula (b1-2).

In the above general formula (b1-2), R⁴¹, R⁴², and R⁴³ each independently represents an alkyl group, an acetyl group, an alkoxy group, a carboxy group, or a hydroxyalkyl group.

The alkyl group for R⁴¹, R⁴², and R⁴³ is preferably a lower alkyl group of 1 to 5 carbon atom, more preferably a linear or branched alkyl group, and still more preferably a methyl group, an ethyl group, a propyl group, an isopropyl group, an n-butyl group, a tert-butyl group, a tert-pentyl group, or an isopentyl group.

The alkoxy group for R⁴¹, R⁴², and R⁴³ is preferably an alkoxy group of 1 to 5 carbon atoms, more preferably a linear or branched alkoxy group, and particularly preferably a methoxy group or an ethoxy group.

The hydroxyalkyl group for R⁴¹, R⁴², and R⁴³ is preferably a group in which one or more hydrogen atoms in the aforementioned alkyl group for R⁴¹, R⁴², and R⁴³ are substituted with hydroxyl groups, and examples thereof include a hydroxymethyl group, a hydroxyethyl group, and a hydroxypropyl group.

n₁ represents an integer of 0 to 3, is preferably 1 or 2, and more preferably 1.

n₂ and n₃ each independently represents an integer of 0 to 3. It is preferable that n₂ and n₃ each be independently 0 or 1, and it is more preferable that both of n₂ and n₃ be 0.

In this regard, however, not all of n₁, n₂, and n₃ are simultaneously 0.

In the general formula (b1-2), X⁻ represents an anion. There is no particular restriction on the anion moiety for X⁻, and any anion moiety can be appropriately used which is known as an anion moiety of an onium salt-based acid generator. For example, an anion represented by the general formula: R¹⁴SO₃⁻) (wherein, R¹⁴ represents a linear, branched or cyclic alkyl group, or a halogenated alkyl group) can be used.

In the general formula: R¹⁴SO₃⁻, R¹⁴ represents a linear, branched or cyclic alkyl group, or a halogenated alkyl group.

The linear or branched alkyl group for R¹⁴ preferably has 1 to 10 carbon atoms, more preferably has 1 to 8 carbon atoms, and most preferably has 1 to 4 carbon atoms.

The cyclic alkyl group for R¹⁴ preferably has 4 to 15 carbon atoms, more preferably has 4 to 10 carbon atoms, and most preferably has 6 to 10 carbon atoms.

R¹⁴ is preferably a halogenated alkyl group. That is, in the formula (b1-2), X⁻ is preferably a halogenated alkylsulfonate ion. The halogenated alkyl group is a group in which a part of or all of hydrogen atoms in the alkyl group are substituted with halogen atoms. Here, as the halogenated alkyl group, the alkyl groups for R¹⁴ in which a part of or all of hydrogen atoms are substituted with halogen atoms can be used. Examples of the halogen atoms which replace the hydrogen atoms include a fluorine atom, a chlorine atom, a bromine atom, and an iodine atom. In the halogenated alkyl group, 50 to 100% of all the hydrogen atoms are preferably substituted with halogen atoms, and it is more preferable that all of the hydrogen atoms be substituted with halogen atoms.

Here, the halogenated alkyl group is preferably a linear, branched, or cyclic fluorinated alkyl group.

The number of carbon atoms in the linear or branched fluorinated alkyl group is preferably 1 to 10, more preferably 1 to 8, and most preferably 1 to 4.

The number of carbon atoms in the cyclic fluorinated alkyl group is preferably 4 to 15, more preferably 4 to 10, and most preferably 6 to 10.

Furthermore, the fluorination rate of the fluorinated alkyl group (proportion of fluorine atoms with which hydrogen atoms are substituted, relative to all hydrogen atoms in the alkyl group before fluorination (hereinafter, referred to as the same)) is preferably within a range of 10 to 100%, more preferably 50 to 100%, and those wherein all hydrogen atoms are substituted with fluorine atoms are most preferable, because the strength of the acid increases.

In the above general formula (b1-2), as X⁻, anions represented by the general formula (b-3) shown below and anions represented by the general formula (b-4) shown below can be used.

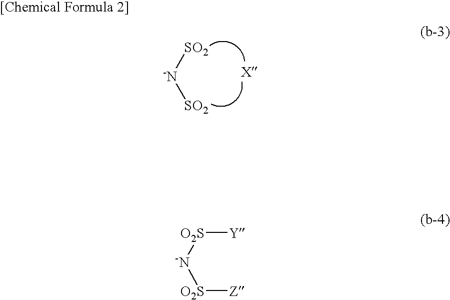

(In the formula, X″ represents an alkylene group of 2 to 6 carbon atoms in which at least one hydrogen atom is substituted with a fluorine atom; and Y″ and Z″ each independently represents an alkyl group of 1 to 10 carbon atoms in which at least one hydrogen atom is substituted with a fluorine atom.)

In the above general formula (b-3), X″ represents a linear or branched alkylene group in which at least one hydrogen atom has been substituted with a fluorine atom, and the alkylene group preferably has 2 to 6 carbon atoms, more preferably 3 to 5 carbon atoms, and most preferably 3 carbon atoms.

In the above general formula (b-4), Y″ and Z″ each independently represents a linear or branched alkyl group in which at least one hydrogen atom has been substituted with a fluorine atom, and the alkyl group preferably has 1 to 10 carbon atoms, more preferably 1 to 7 carbon atoms, and most preferably 1 to 3 carbon atoms.

Within the above range of carbon atoms, lower numbers of carbon atoms of the alkylene group for X″ or the alkyl groups for Y″ and Z″ result in better solubility within the resist solvent, and are consequently preferred.

Furthermore, in the alkylene group for X″ or the alkyl groups for Y″ and Z″, higher numbers of hydrogen atoms that have been substituted with fluorine atoms results in increasing the strength of an acid and also improving the transparency relative to high energy light beams of 200 nm or less, or electron beams, and is consequently preferred. The proportion of fluorine atoms in the above alkylene group or alkyl group is preferably within the range of 70 to 100%, and more preferably 90 to 100%. A perfluoroalkylene group or perfluoroalkyl group wherein all hydrogen atoms are substituted with fluorine atoms is most preferable.

Specific examples suitable as the compound of the first aspect of the present invention include the following.

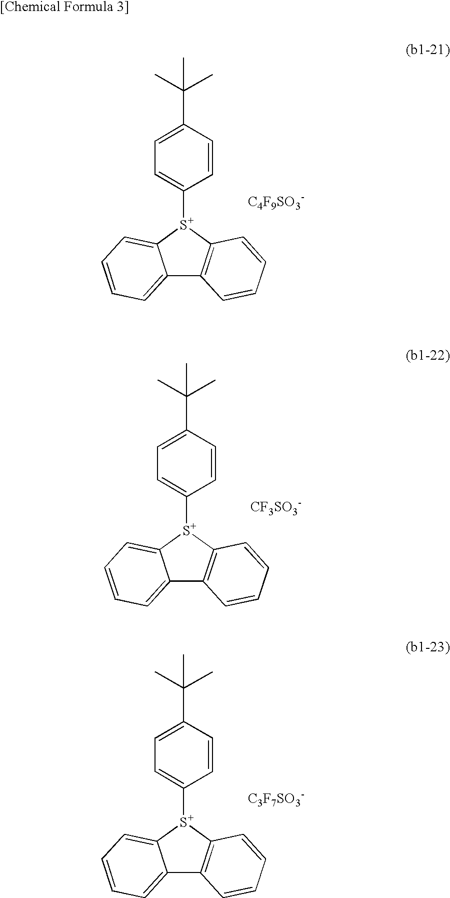

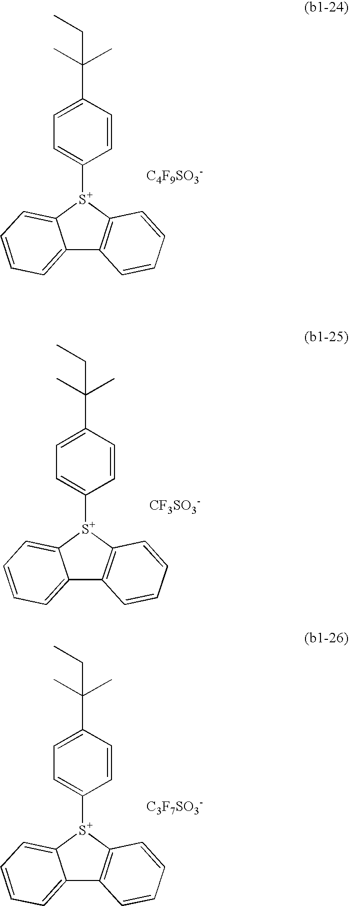

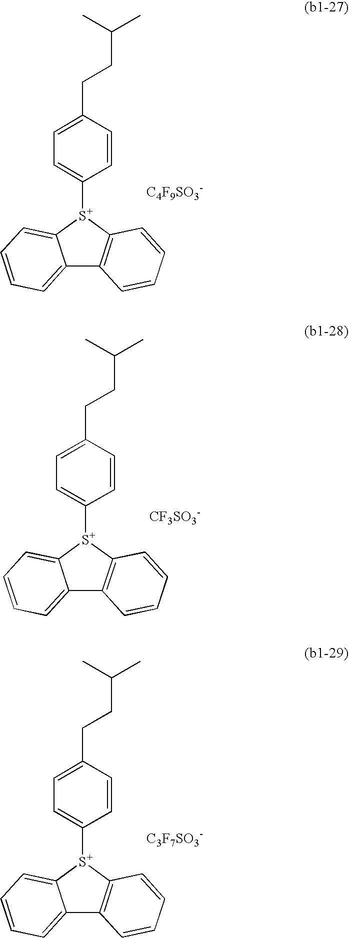

Of these, the compound represented by the above formula (b1-21) or (b1-24) is preferable.

### <Method of Manufacturing Compound>

The compound (b1-2) according to the first aspect of the present invention can be obtained, for example, by reacting a compound represented by the general formula (b1-0-21) shown below and a compound represented by the general formula (b1-0-22) shown below in a solvent such as chlorobenzene and iodobenzene using a catalyst such as copper benzoate (II) at 80° C. to 130° C., and more preferably at 100° C. to 120° C., for 0.5 to 3 hours, and more preferably for 1 to 2 hours.

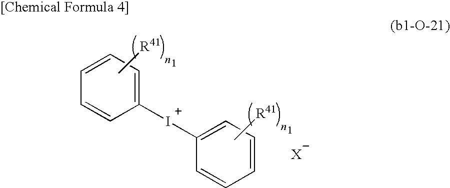

(In the formula, R⁴¹ is the same as R⁴¹ described above in the formula (b1-2); n₁ is the same as n₁ described above in the formula (b1-2); and X⁻ is the same as X⁻ described above in the formula (b1-2).)

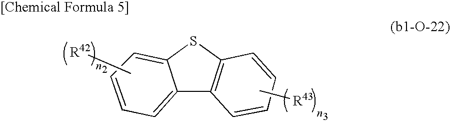

(In the formula, R⁴² and R⁴³ are respectively the same as R⁴² and R⁴³ described above in the formula (b1-2); n₂ and n₃ are respectively the same as n₂ and n₃ described above in the formula (b1-2), where not all of n₁, n₂, and n₃ in the above two formulae are simultaneously 0.)

### <<Acid Generator>>

The acid generator (hereinafter, sometimes referred to as acid generator (B1)) according to the second aspect of the present invention is composed of a compound represented by the aforementioned general formula (b1-2). In the formula, R⁴¹, R⁴², R⁴³, n₁, n₂, n₃, and X⁻ are the same as those described in the compound of the first aspect of the present invention.

### <<Resist Composition>>

The resist composition of the third aspect of the present invention includes a base component (A) (hereinafter, referred to as component (A)) which exhibits changed solubility in an alkali solution under action of acid and an acid generator component (B) (hereinafter, referred to as component (B)) which generates an acid upon exposure, wherein the component (B) includes the acid generator (B1) composed of the compound represented by the above general formula (b1-2).

In the resist composition of the present invention, a polymer material which exhibits changed solubility in an alkali developing solution under action of acid can be used as the component (A), and a low molecular material which exhibits changed solubility in an alkali developing solution under action of acid can also be used as the component (A).

Also, the resist composition of the present invention may be a negative resist composition or a positive resist composition.

In the case that the resist composition of the present invention is a negative resist composition, for example, the component (A) is an alkali-soluble resin, and a cross-linking agent (C) is blended with the resist composition.

In the negative resist composition, when an acid is generated from the component (B) upon exposure during resist pattern formation, the action of this acid causes cross-linking between the alkali-soluble resin and the cross-linking agent, and the exposed portion becomes alkali-insoluble.

As the alkali-soluble resin, it is preferable to use a resin having a structural unit derived from at least one of an α-(hydroxyalkyl)acrylic acid and a lower alkyl ester of α-(hydroxyalkyl)acrylic acid, because it enables formation of a satisfactory resist pattern with minimal swelling. Here, the term “α-(hydroxyalkyl) acrylic acid” means one or both of an acrylic acid in which a hydrogen atom is bonded with the carbon atom at the α-position with which the carboxyl group bonded, and an α-hydroxyalkylacrylic acid in which a hydroxyalkyl group (preferably a hydroxyalkyl group of 1 to 5 carbon atoms) is bonded with the carbon atom at the α-position.

As the cross-linking agent (C), typically, an amino-based cross-linking agent such as a glycoluril having a methylol group or alkoxymethyl group is preferable, as it enables formation of a satisfactory resist pattern with minimal swelling. The amount of the cross-linking agent (C) added is preferably within the range of 1 to 50 parts by weight, relative to 100 parts by weight of the alkali-soluble resin.

In the case that the resist composition of the present invention is a positive resist composition, the component (A) should be an alkali-insoluble one which contains an acid dissociable, dissolution inhibiting group, and exhibits increased solubility in an alkali developing solution under action of acid when an acid is generated from the component (B) upon exposure. Therefore, in the formation of a resist pattern, when a resist film obtained by applying the positive resist composition on the substrate is subjected to selective exposure, the exposed area becomes soluble in an alkali, while the unexposed area remains alkali-insoluble, and hence a resist pattern can be formed by a developing treatment with an alkali.

In the resist composition of the present invention, the component (A) is preferably a base component which exhibits increased solubility in an alkali developing solution under action of acid, and more preferably a resin component (A1) (hereinafter, referred to as component (A1)) which exhibits increased solubility in an alkali developing solution under action of acid. That is, the resist composition of the present invention is preferably a positive resist composition. Also, the resist composition of the present invention can be preferably used as a resist composition for immersion lithography in a method of forming a resist pattern including immersion exposure. Further, the resist composition of the present invention can be preferably used as a positive resist composition for forming an upper-layer resist film in a method of forming a resist pattern including the formation of a triple-layer resist laminate.

Subsequently, in a method of forming a resist pattern including immersion exposure and/or formation of a triple-layer resist laminate, the component (A1), suitably used in a positive resist composition, will be described in more detail.

### <Component (A1)>

The component (A1) suitably used in the positive resist composition preferably includes a structural unit (a1) derived from an acrylate ester having an acid dissociable, dissolution inhibiting group.

Further, the component (A1) preferably includes a structural unit (a2) derived from an acrylate ester having a lactone-containing cyclic group.

Furthermore, the component (A1) preferably includes a structural unit (a3) derived from an acrylate ester having a polar group-containing aliphatic hydrocarbon group.

Here, the term “structural unit derived from an acrylate ester” in the present specification and claims represents a structural unit formed by cleavage of the ethylenic double bond of an acrylate ester.

The term “acrylate ester” is a concept containing an acrylate ester in which a hydrogen atom is bonded with the carbon atom at the α-position, and an α-substituted acrylate ester in which a hydrogen atom bonded with the carbon atom at the α-position is substituted with another substituent group (an atom or group other than a hydrogen atom).

Examples of the substituent group include a halogen atom, a lower alkyl group, and a halogenated lower alkyl group. Examples of the halogen atoms include a fluorine atom, a chlorine atom, a bromine atom and an iodine atom. Of these, a fluorine atom is preferable. The term “halogenated lower alkyl group” is a group in which at least one of or all of hydrogen atoms in the above lower alkyl group are substituted with halogen atoms.

The term “α-position (carbon atom at the α-position)” in a structural unit derived from an acrylate ester means a carbon atom with which a carbonyl group is bonded, if not otherwise specified.

In the acrylate ester, specific examples of the lower alkyl group as the substituent group at the α-position include linear or branched lower alkyl groups such as a methyl group, an ethyl group, a propyl group, an isopropyl group, an n-butyl group, an isobutyl group, a tert-butyl group, a pentyl group, an isopentyl group, or a neopentyl group.

In the present invention, the group which is bonded with the α-position is preferably a hydrogen atom, a halogen atom, a lower alkyl group, or a halogenated lower alkyl group, more preferably a hydrogen atom, a fluorine atom, a lower alkyl group, or a fluorinated lower alkyl group, and still more preferably a hydrogen atom or a methyl group, in terms of industrial availability.

Structural Unit (a1)

Structural unit (a1) is a structural unit derived from an acrylate ester having an acid dissociable, dissolution inhibiting group.

As the acid dissociable, dissolution inhibiting group in the structural unit (a1), any of the groups that have been proposed as acid dissociable, dissolution inhibiting groups for the base resins of chemically amplified resists can be used, provided the group has an alkali dissolution-inhibiting effect that renders the entire component (A1) alkali-insoluble prior to dissociation, and then following dissociation by action of acid, causes the entire component (A1) to change to an alkali-soluble state.

Generally, groups that form either a cyclic or chain-like tertiary alkyl ester with the carboxyl group of the (meth)acrylic acid or the like; and acetal-type acid dissociable, dissolution inhibiting groups such as alkoxyalkyl groups are widely known. Here, the term “(meth)acrylic acid” means one or both of acrylic acid and methacrylic acid.

Here, the term “tertiary alkyl ester” means a structure in which an ester is formed by substituting the hydrogen atom of a carboxyl group with a chain-like or cyclic alkyl group, and a tertiary carbon atom within the chain-like or cyclic alkyl group is bonded with the oxygen atom at the terminal of the carbonyloxy group (—C(O)—O—). In the tertiary alkyl ester, the bond of the oxygen atom with the tertiary carbon atom is cleaved by the action of acid.

Here, the chain-like or cyclic alkyl group may contain a substituent group.

Hereinafter, for the sake of simplicity, groups that exhibit acid dissociability as a result of the formation of a tertiary alkyl ester with a carboxyl group are referred to as “tertiary alkyl ester-type acid dissociable, dissolution inhibiting groups”.

Examples of the tertiary alkyl ester-type acid dissociable, dissolution inhibiting groups include aliphatic branched, acid dissociable, dissolution inhibiting groups and aliphatic cyclic group-containing acid dissociable, dissolution inhibiting groups.

Here, the term “aliphatic” is a relative concept used in relation to the term “aromatic”, and defines a group, compound or the like that contains no aromaticity.

The term “aliphatic branched” means a branched structure having no aromaticity.

The “aliphatic branched, acid dissociable, dissolution inhibiting group” is not limited to groups (hydrocarbon groups) composed of carbon atoms and hydrogen atoms, and is preferably a hydrocarbon group.

Further, the “hydrocarbon group” may be either saturated or unsaturated, and is preferably saturated.

Examples of aliphatic branched, acid dissociable, dissolution inhibiting groups include tertiary alkyl groups of 4 to 8 carbon atoms, and specific examples include a tert-butyl group, a tert-pentyl group and a tert-heptyl group.

The term “aliphatic cyclic group (alicyclic group)” means a monocyclic or polycyclic group which has no aromaticity.

The “aliphatic cyclic group” within the structural unit (a1) may or may not contain a substituent group. Examples of substituent groups include a lower alkyl group of 1 to 5 carbon atoms, a fluorine atom, a fluorinated lower alkyl group of 1 to 5 carbon atoms, and an oxygen atom (═O).

The basic ring of the “aliphatic cyclic group” exclusive of substituent groups is not limited to groups (hydrocarbon groups) composed of carbon atoms and hydrogen atoms, and is preferably a hydrocarbon group. Further, the “hydrocarbon group” may be either saturated or unsaturated, and is preferably saturated. The “aliphatic cyclic group” is preferably a polycyclic group.

Examples of the aliphatic cyclic groups include groups in which one or more hydrogen atoms have been removed from a mono cycloalkane or a polycycloalkane such as a bicycloalkane, tricycloalkane or tetracycloalkane in which a lower alkyl group, a fluorine atom or a fluorinated alkyl group may or may not be included as a substituent group. Specific examples thereof include groups in which one or more hydrogen atoms have been removed from a monocycloalkane such as cyclopentane and cyclohexane, and a polycycloalkane such as adamantane, norbornane, isobornane, tricyclodecane, and tetracyclododecane.

As the aliphatic cyclic group-containing acid dissociable, dissolution inhibiting group, for example, a group which has a tertiary carbon atom on the ring structure of the cycloalkyl group can be used. Specific examples thereof include a 2-methyl-2-adamantyl group and a 2-ethyl-2-adamantyl group. Also, groups having an aliphatic cyclic group such as an adamantyl group, and a branched alkylene group having a tertiary carbon atom bonded thereto, as in a structural unit represented by the general formula (a1″) shown below, can be used.

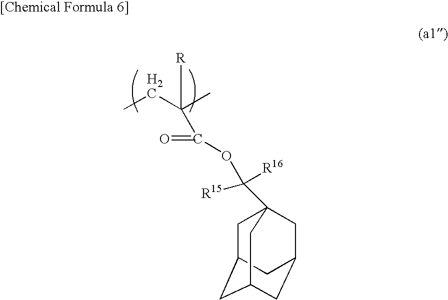

(In the formula, R represents a hydrogen atom, a halogen atom, a lower alkyl group, or a halogenated lower alkyl group; and R¹⁵ and R¹⁶ each independently represents an alkyl group (wherein, the alkyl group may be linear or branched, and preferably has 1 to 5 carbon 5 atoms).)

In the above formula, the halogen atom, lower alkyl group, or halogenated lower alkyl group for R is the same as the halogen atom, lower alkyl group, or halogenated lower alkyl group which may be bonded with the α-position of the above-mentioned acrylate ester.

An “acetal-type acid dissociable, dissolution inhibiting group” generally replaces a hydrogen atom at the terminal of an alkali-soluble group such as a carboxy group or a hydroxyl group, so as to be bonded with an oxygen atom. When acid is generated upon exposure, the generated acid acts to break the bond between the acetal-type acid dissociable, dissolution inhibiting group and the oxygen atom with which the acetal-type, acid dissociable, dissolution inhibiting group is bonded.

Examples of the acetal-type acid dissociable, dissolution inhibiting groups include groups represented by the general formula (p1) shown below.

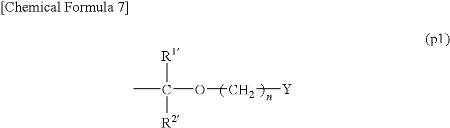

(In the formula, R¹′ and R²′ each independently represents a hydrogen atom or a lower alkyl group; n represents an integer of 0 to3; and Y represents a lower alkyl group or an aliphatic cyclic group.)

In the above formula, n is preferably an integer of 0 to 2, more preferably 0 or 1, and most preferably 0.

As the lower alkyl group for R¹′ or R²′, the same lower alkyl groups as those described above in R can be used. As the lower alkyl group of R¹′ or R²′, a methyl group or an ethyl group is preferable, and a methyl group is most preferable.

In the present invention, at least one of R¹′ and R²′ is preferably a hydrogen atom. That is, it is preferable that the acid dissociable, dissolution inhibiting group (p1) be a group represented by the general formula (p1-1) shown below.

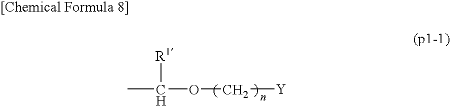

(In the formula, R¹′, n, and Y are respectively the same as R¹′, n, and Y described above in the general formula (p1).

As the lower alkyl group for Y, the same lower alkyl group as those described above in R can be used.

As the aliphatic cyclic group for Y, any of the aliphatic monocyclic or polycyclic groups which have been proposed for conventional ArF resists and the like can be used by being appropriately selected. For example, the same groups described above in the “aliphatic cyclic group” can be used.

Further, as the acetal-type, acid dissociable, dissolution inhibiting group, groups represented by the general formula (p2) shown below can also be used.

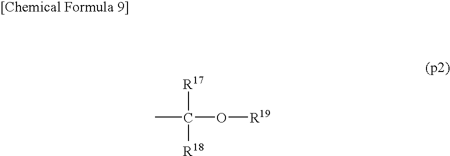

(In the formula, R¹⁷ and R¹⁸ each independently represents a linear or branched alkyl group or a hydrogen atom; and R¹⁹ represents a linear, branched or cyclic alkyl group. Alternately, R¹⁷ and R¹⁹ each independently may represent a linear or branched alkylene group, wherein the terminal of R¹⁷ is bonded with the terminal of R¹⁹ thereby forming a ring.)

The alkyl group for R¹⁷ and R¹⁸ preferably has 1 to 15 carbon atoms, and may be either linear or branched. As the alkyl group, an ethyl group or a methyl group is preferable, and a methyl group is most preferable. It is particularly preferable that either one of R¹⁷ and R¹⁸ be a hydrogen atom, and the other be a methyl group.

R¹⁹ represents a linear, branched or cyclic alkyl group which preferably has 1 to 15 carbon atoms, and may be any of linear, branched or cyclic.

When R¹⁹ represents a linear or branched alkyl group, it is preferably an alkyl group of 1 to 5 carbon atoms, more preferably an ethyl group or methyl group, and most preferably an ethyl group.

When R¹⁹ represents a cyclic alkyl group, it preferably has 4 to 15 carbon atoms, more preferably 4 to 12 carbon atoms, and most preferably 5 to 10 carbon atoms. Specific examples of the cyclic alkyl group include groups in which one or more hydrogen atoms have been removed from a monocycloalkane or a polycycloalkane such as a bicycloalkane, tricycloalkane or tetracycloalkane, in which a fluorine atom or a fluorinated alkyl group may or may not be included as a substituent group. Specific examples thereof include groups in which one or more hydrogen atoms have been removed from a monocycloalkane such as cyclopentane and cyclohexane, and a polycycloalkane such as adamantane, norbornane, isobornane, tricyclodecane, and tetracyclododecane. Of these, a group in which one or more hydrogen atoms have been removed from adamantane is preferable.

In the general formula (p2), R¹⁷ and R¹⁹ may each independently represent a linear or branched alkylene group (preferably an alkylene group of 1 to 5 carbon atoms), where the terminal of R¹⁹ is bonded with the terminal of R¹⁷.

In such a case, a cyclic group is formed by R¹⁷, R¹⁹, the oxygen atom with which R¹⁹ is bonded, and the carbon atom with which the oxygen atom and R¹⁷ are bonded. Such a cyclic group is preferably a 4- to 7-membered ring, and more preferably a 4- to 6-membered ring. Specific examples of the cyclic group include a tetrahydropyranyl group and a tetrahydrofuranyl group.

As the structural unit (a1), it is preferable to use at least one member selected from the group consisting of structural units represented by the general formula (a1-0-1) shown below and structural units represented by the general formula (a1-0-2) shown below.

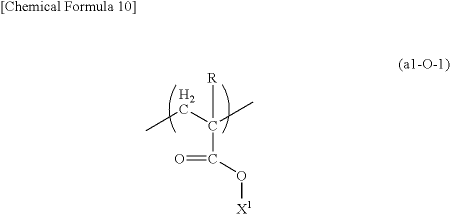

(In the formula, R represents a hydrogen atom, a lower alkyl group, or a halogenated lower alkyl group; and X¹ represents an acid dissociable, dissolution inhibiting group.)

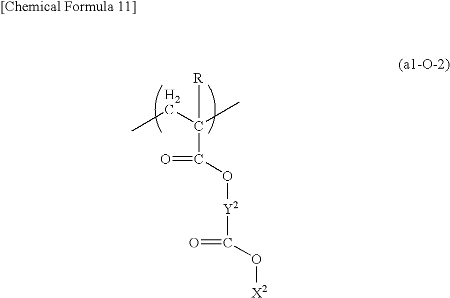

(In the formula, R represents a hydrogen atom, a halogen atom, a lower alkyl group or a halogenated lower alkyl group; X² represents an acid dissociable, dissolution inhibiting group; and Y² represents an alkylene group or an aliphatic cyclic group.)

In the general formula (a1-0-1), the halogen atom, lower alkyl group or halogenated lower alkyl group for R are the same as the halogen atom, lower alkyl group or halogenated lower alkyl group which may be bonded with the α-position of the aforementioned acrylate ester.

X¹ is not particularly limited as long as it is an acid dissociable, dissolution inhibiting group. Examples thereof include the aforementioned tertiary alkyl ester-type acid dissociable, dissolution inhibiting groups and acetal-type acid dissociable, dissolution inhibiting groups, and tertiary alkyl ester-type acid dissociable, dissolution inhibiting groups are preferable.

In the general formula (a1-0-2), R is the same as R described above in the general formula (a1-0-1).

X² is the same as X¹ described above in the general formula (a1-0-1).

Y² is preferably an alkylene group of 1 to 4 carbon atoms or a divalent aliphatic cyclic group. As the aliphatic cyclic group, the same groups as those described above in the explanation of “aliphatic cyclic group” can be used, except that two hydrogen atoms have been removed therefrom.

Specific examples of the structural unit (a1) include structural units represented by the general formulae (a1-1) to (a1-4) shown below.

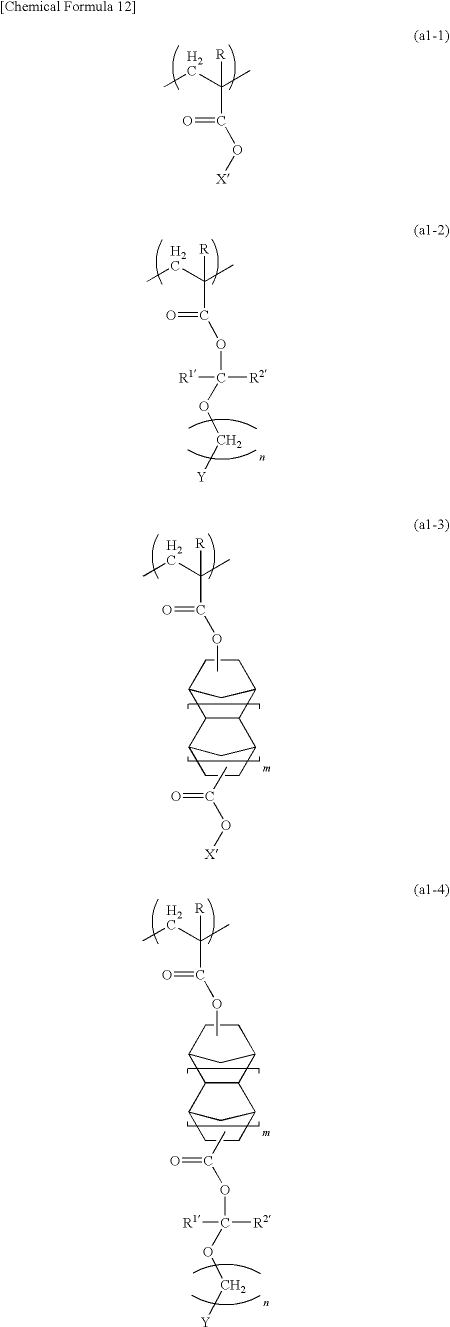

(In the above formulae, X′ represents a tertiary alkyl ester-type acid dissociable, dissolution inhibiting group; Y represents a lower alkyl group of 1 to 5 carbon atoms, or an aliphatic cyclic group; n represents an integer of 0 to 3; m represents an integer of 0 or 1; R represents a hydrogen atom, a halogen atom, a lower alkyl group, or a halogenated lower alkyl group; R¹′ and R²′ each independently represents a hydrogen atom or a lower alkyl group of 1 to 5 carbon atoms.)

In the above formulae (a1-1) to (a1-4), R is the same as R described above in the general formulae (a1-0-1) to (a1-0-2).

It is preferable that at least one of R¹′ and R²′ represent a hydrogen atom, and it is more preferable that both of R¹′ and R²′ represent hydrogen atoms. n is preferably 0 or 1.

The tertiary alkyl ester-type acid dissociable, dissolution inhibiting group for X′ are the same as the above-mentioned tertiary alkyl ester-type acid dissociable, dissolution inhibiting groups for X¹.

Examples of the aliphatic cyclic group for Y include the same groups as those described above in the explanation of “aliphatic cyclic group”.

Specific examples of structural units represented by the general formulae (a1-1) to (a1-4) shown above include the following.

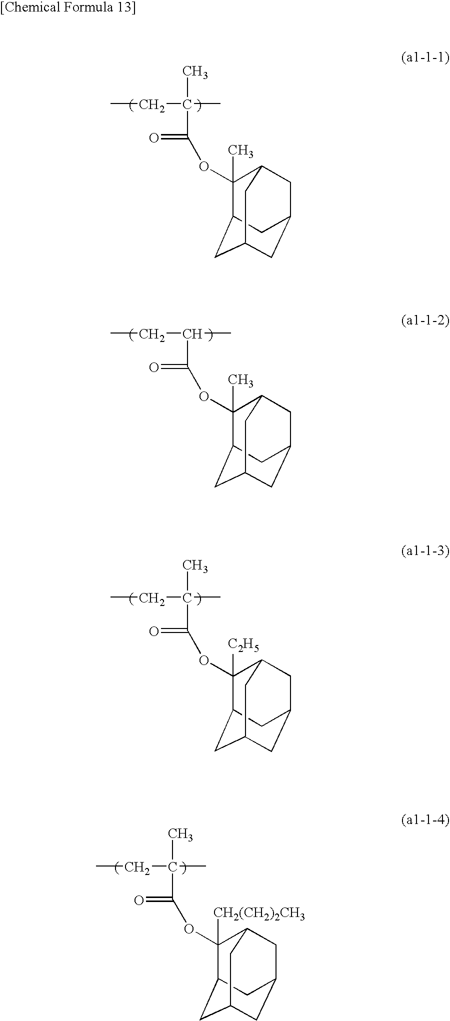

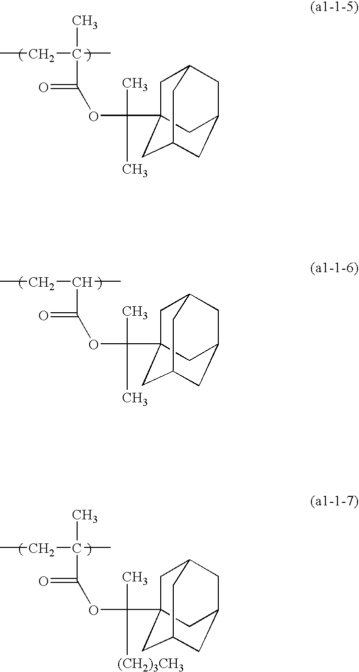

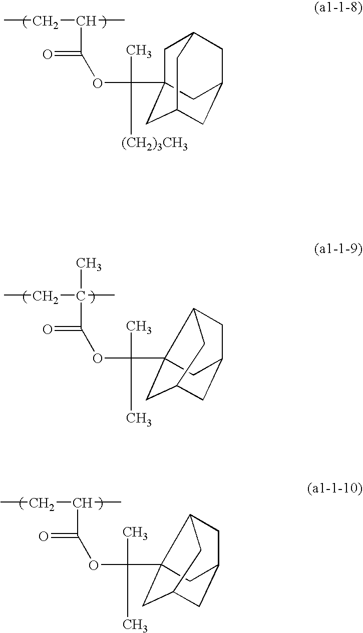

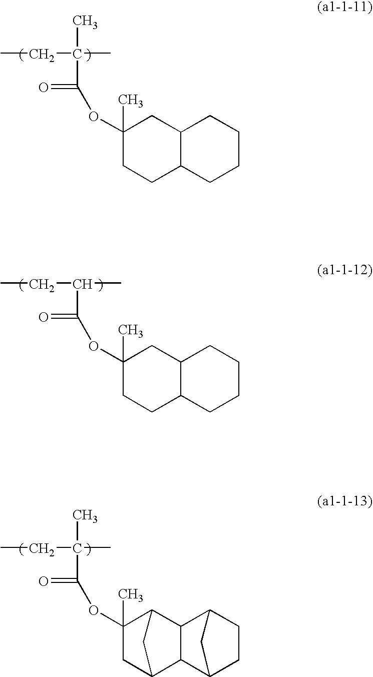

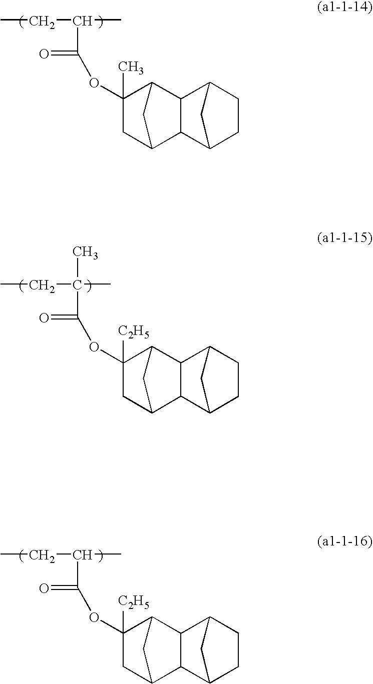

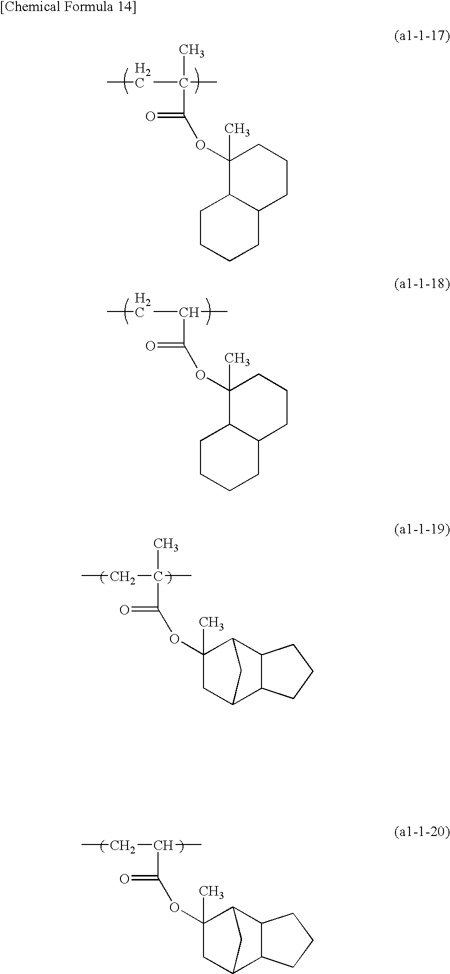

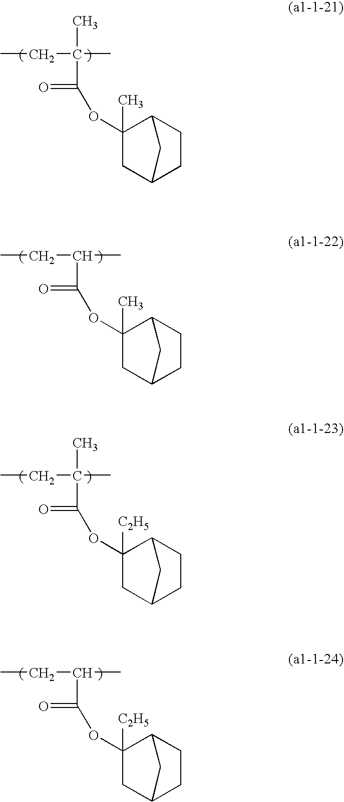

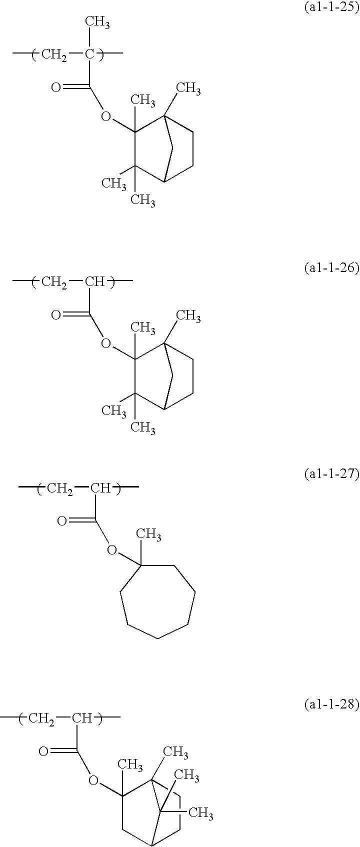

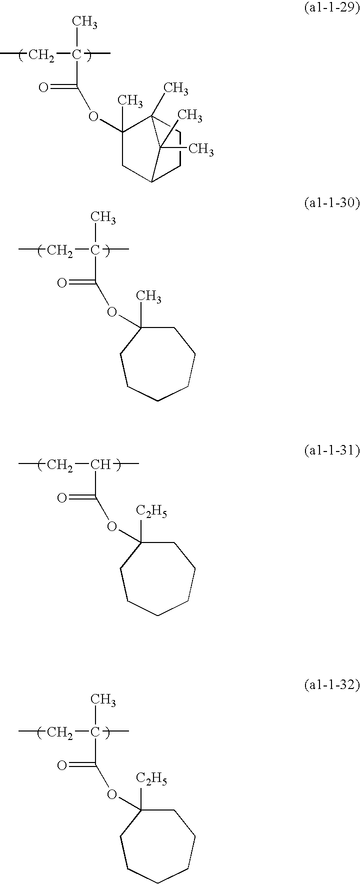

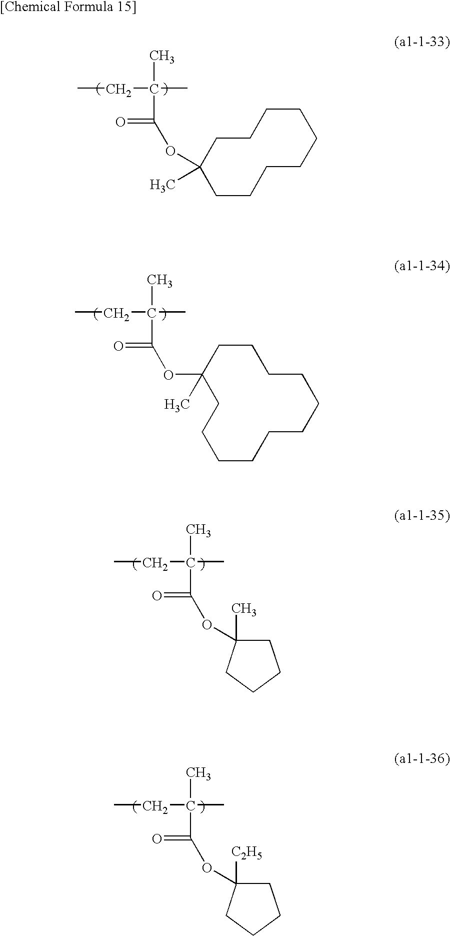

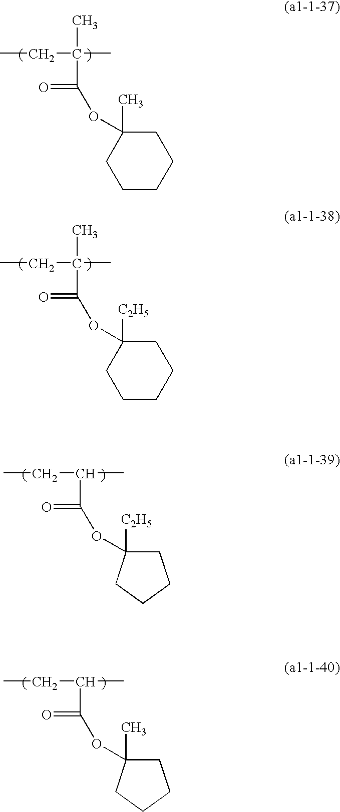

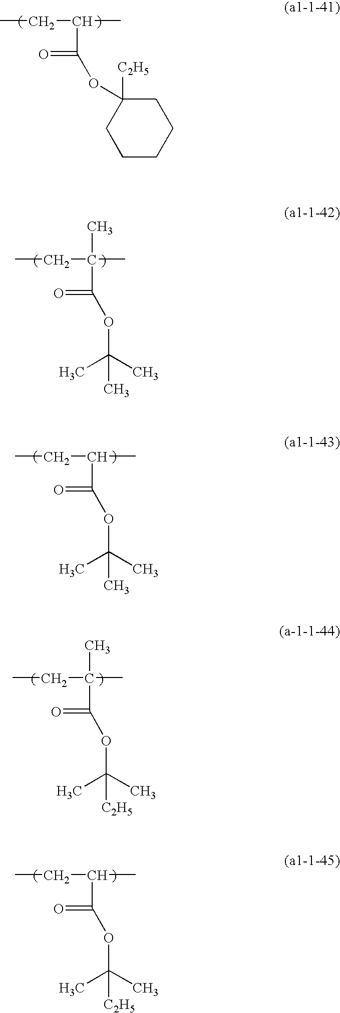

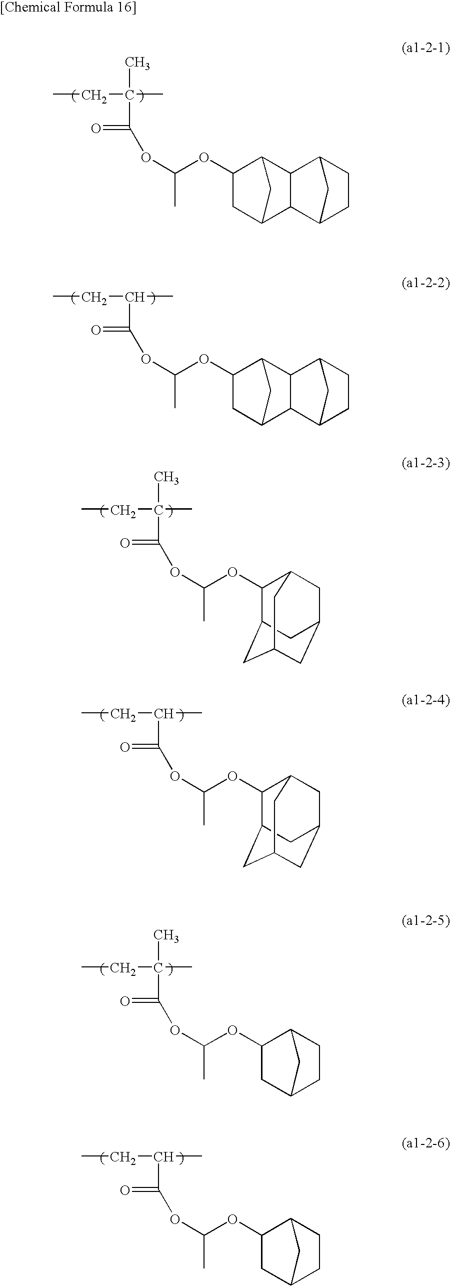

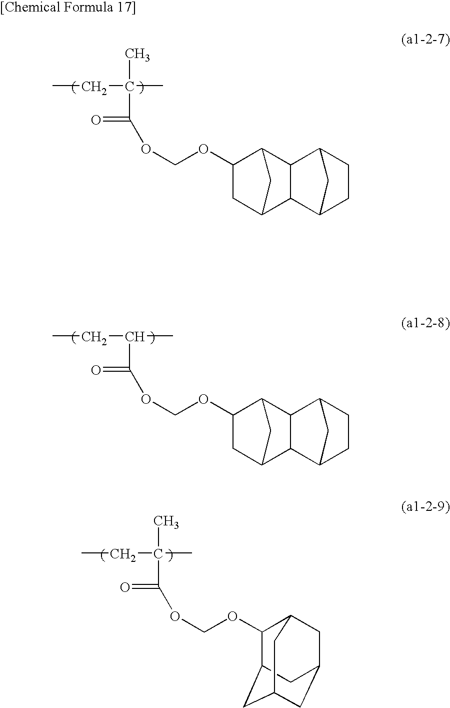

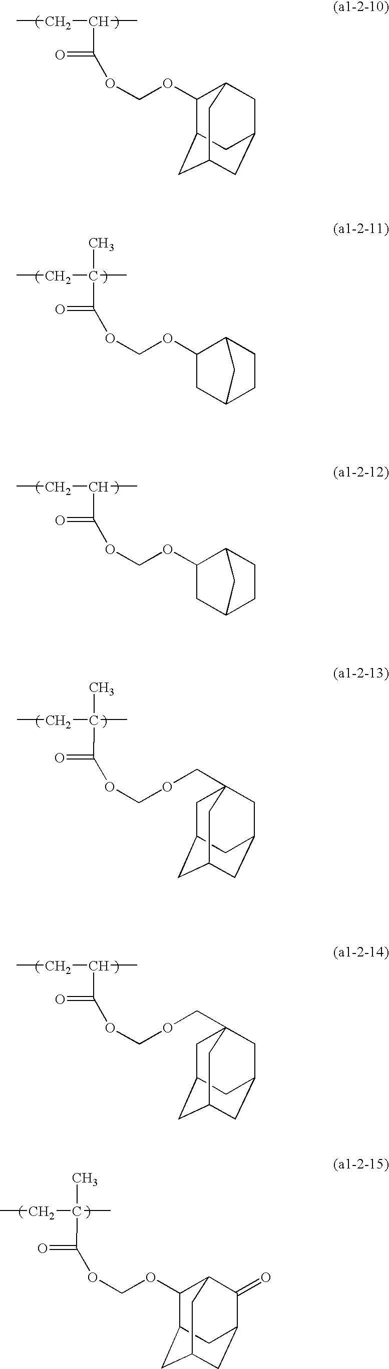

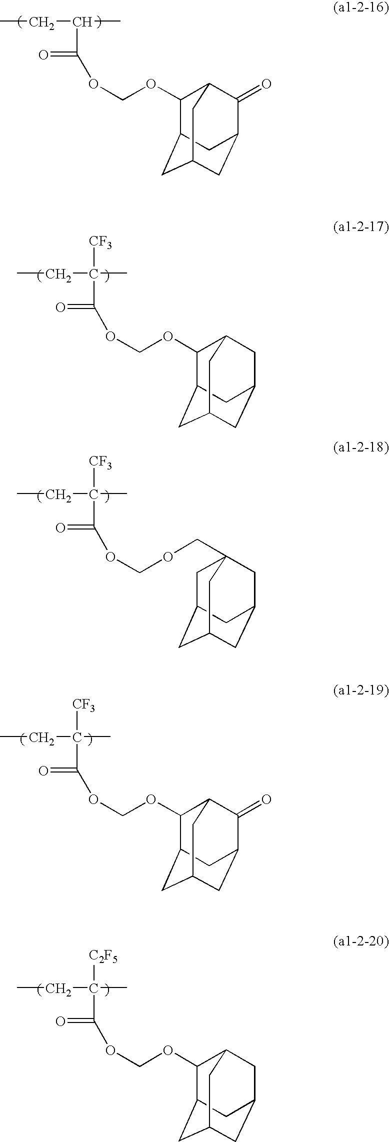

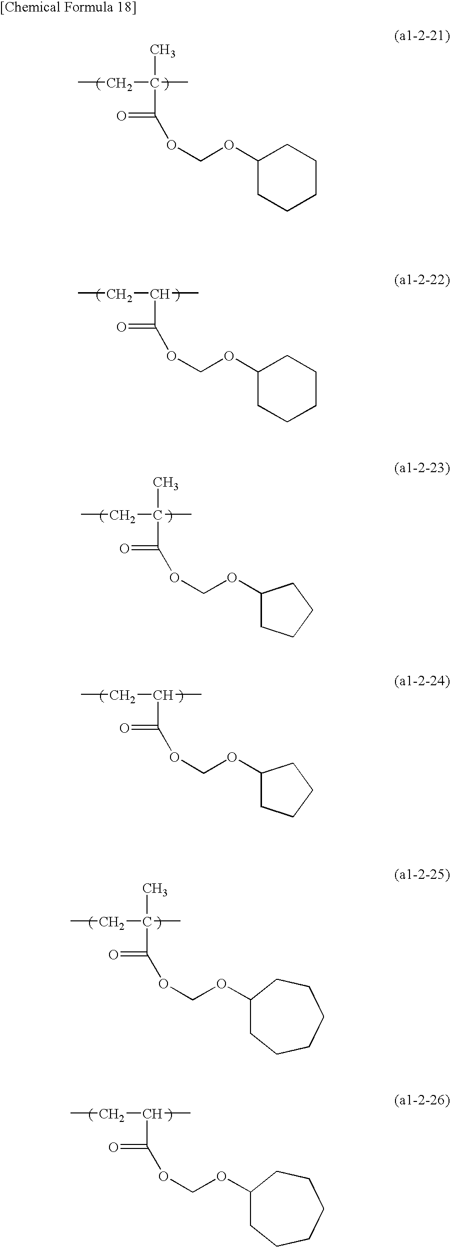

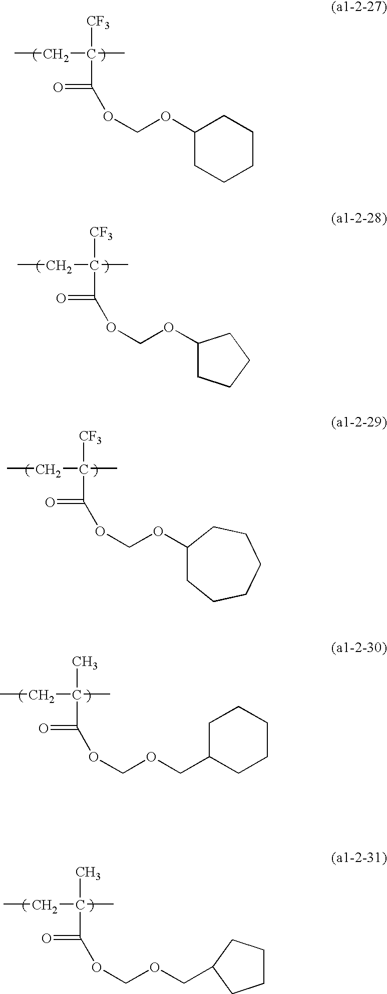

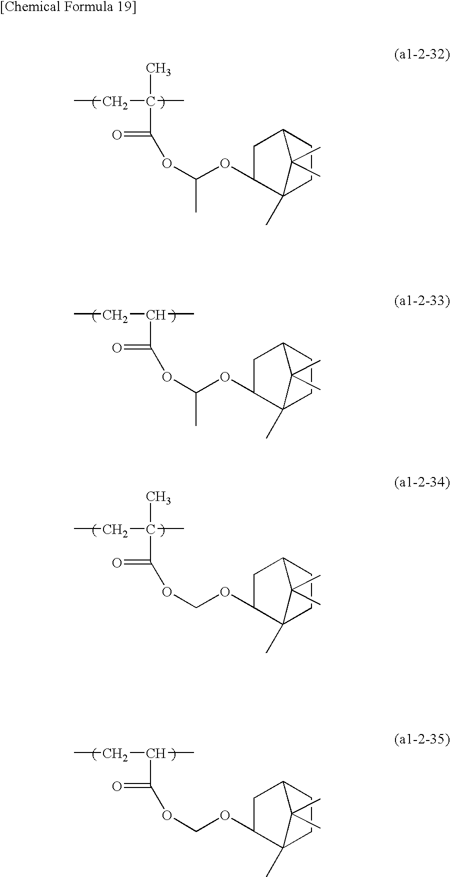

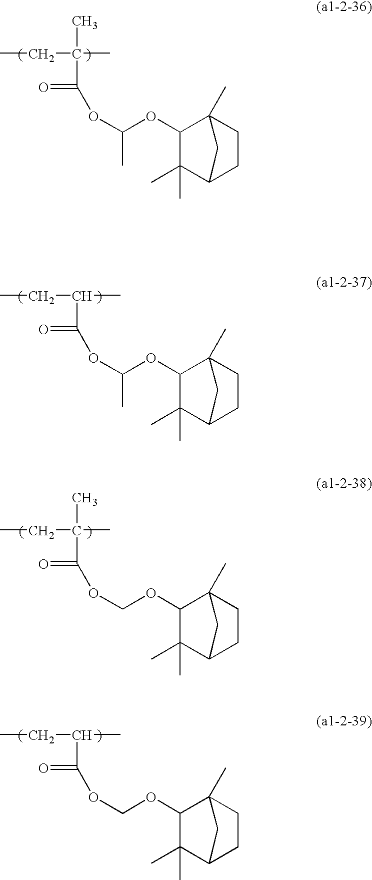

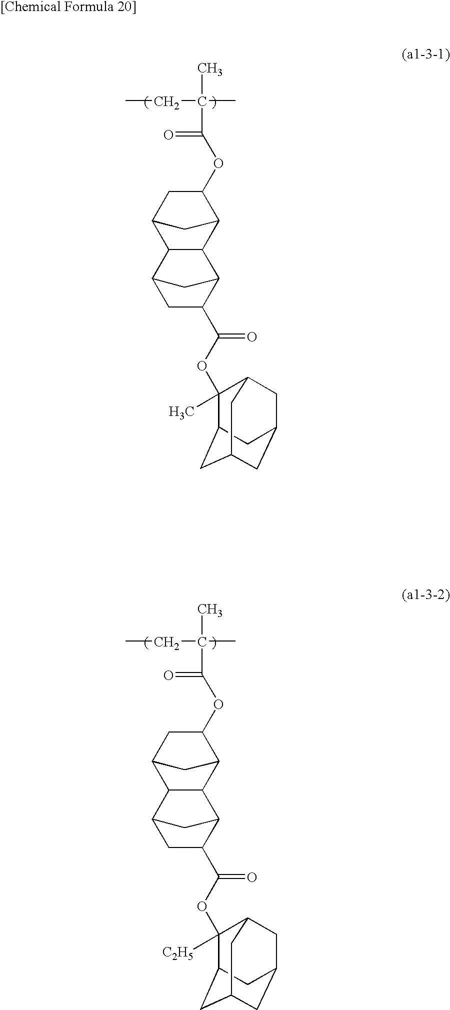

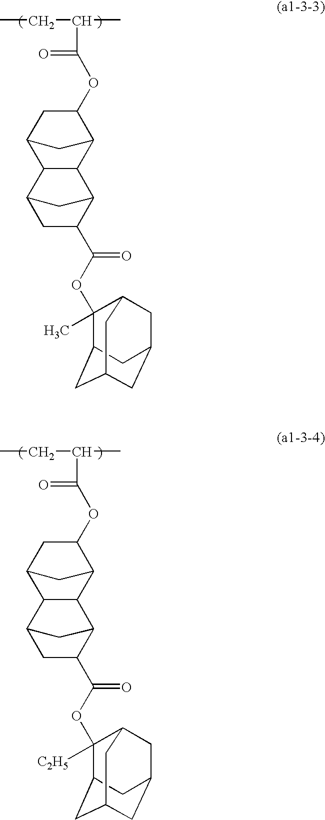

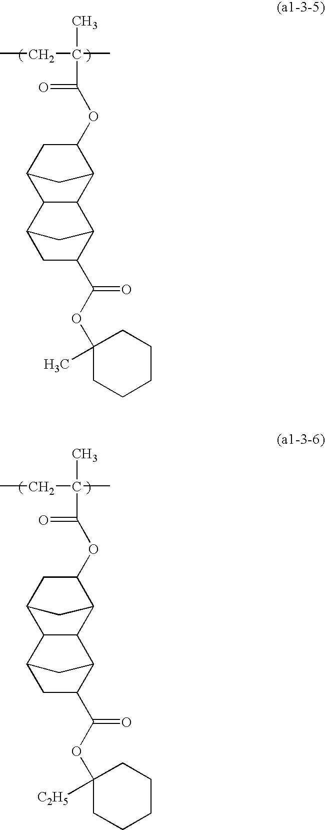

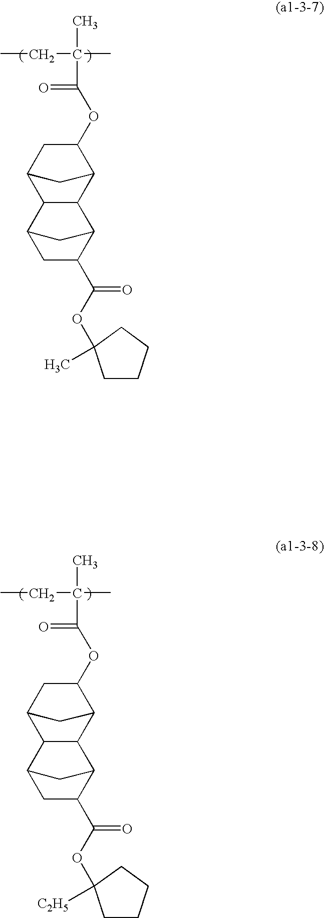

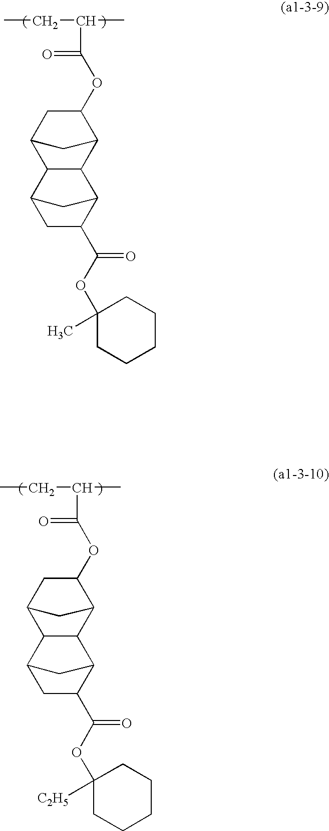

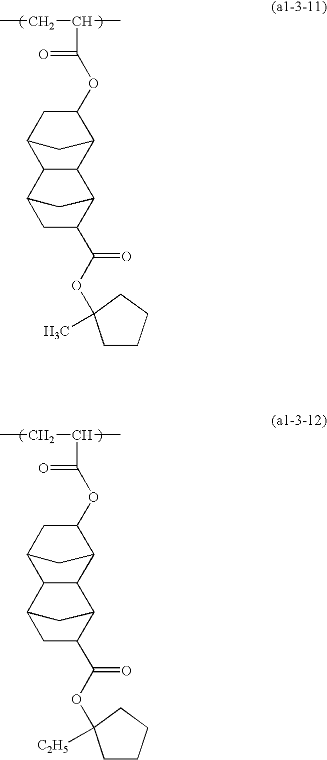

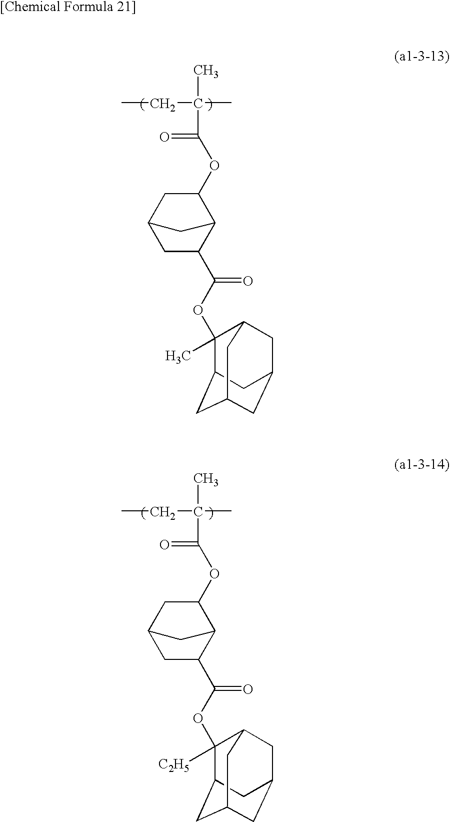

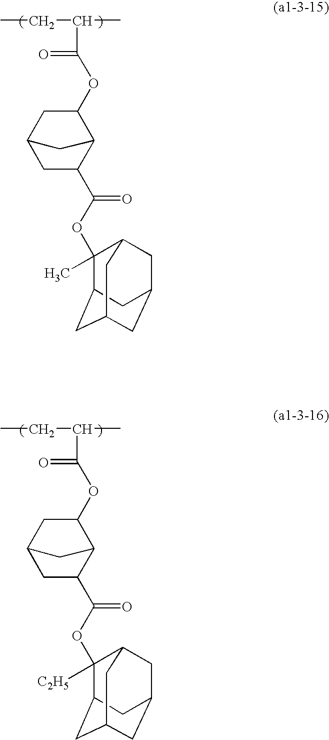

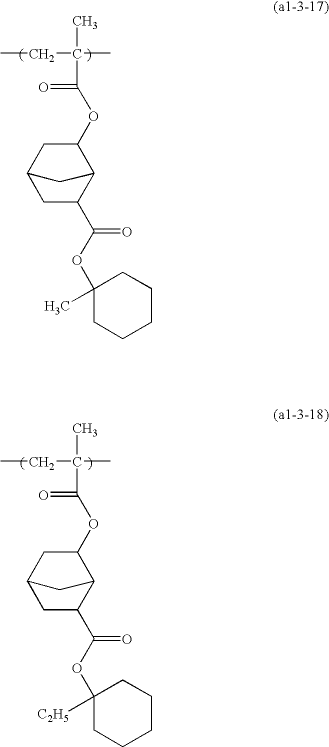

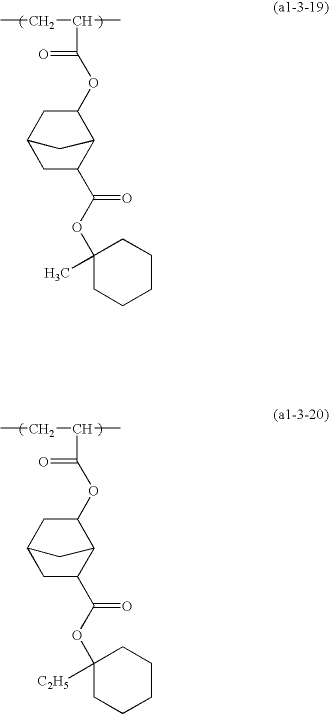

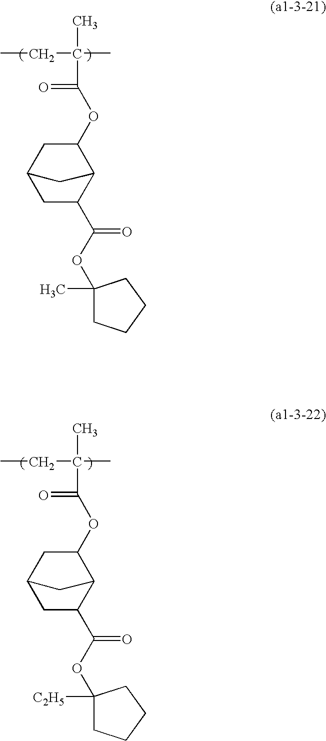

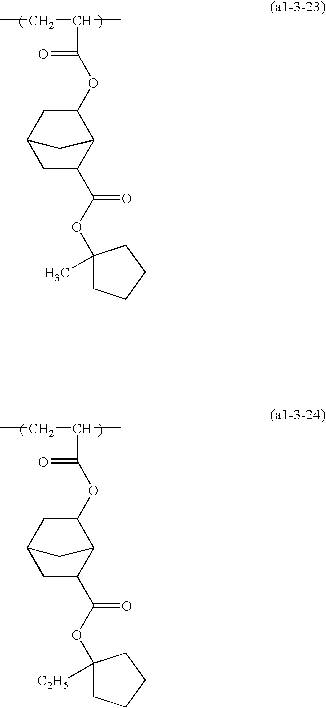

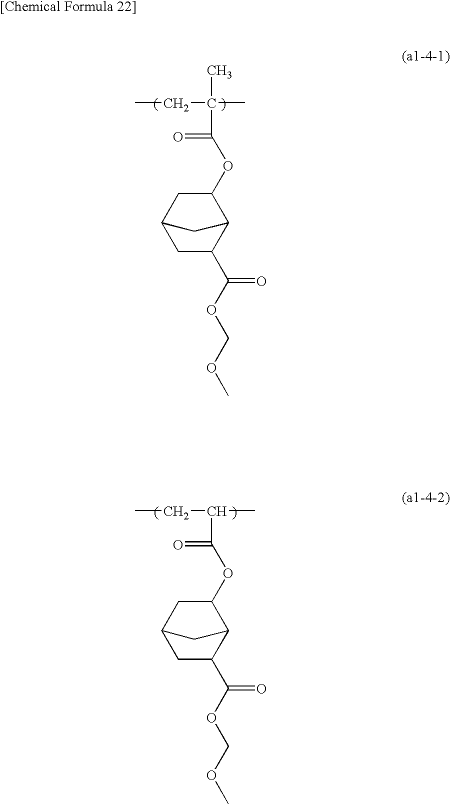

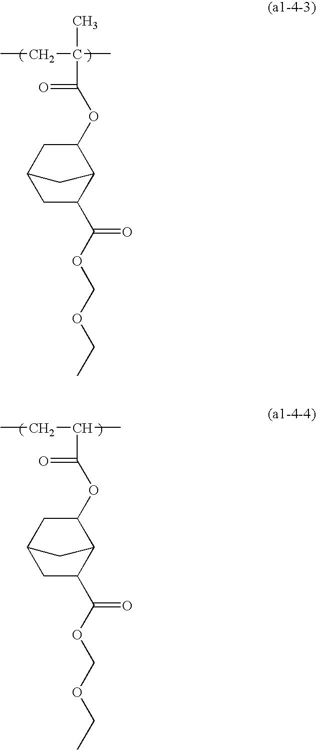

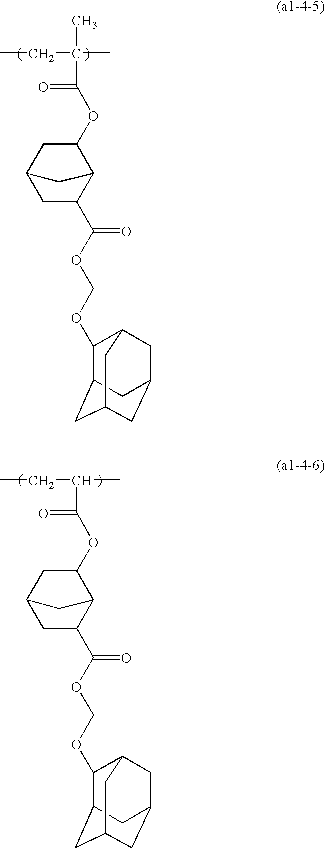

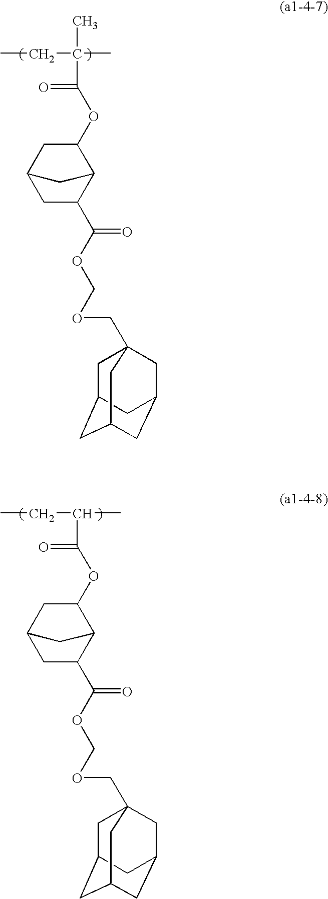

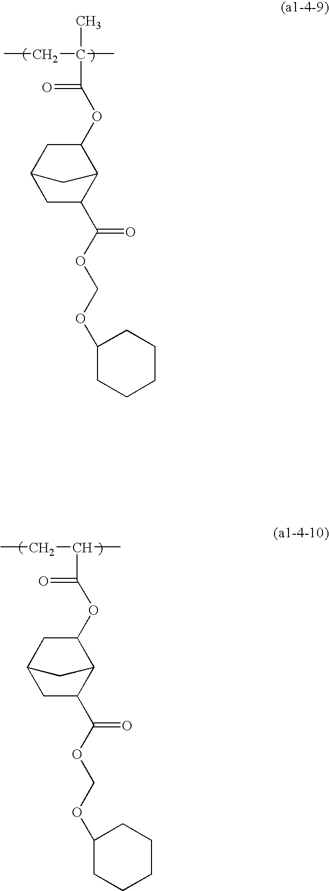

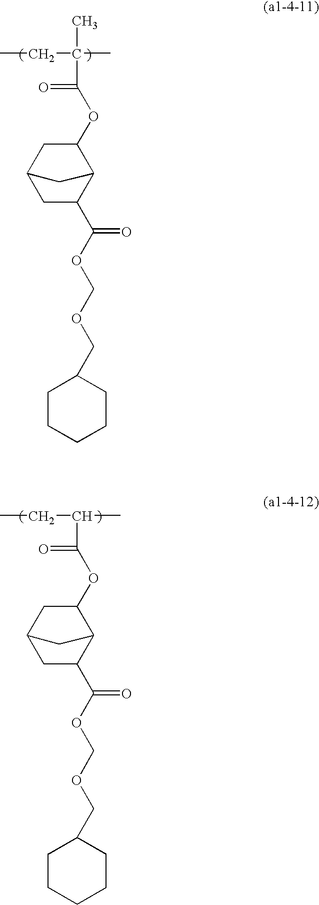

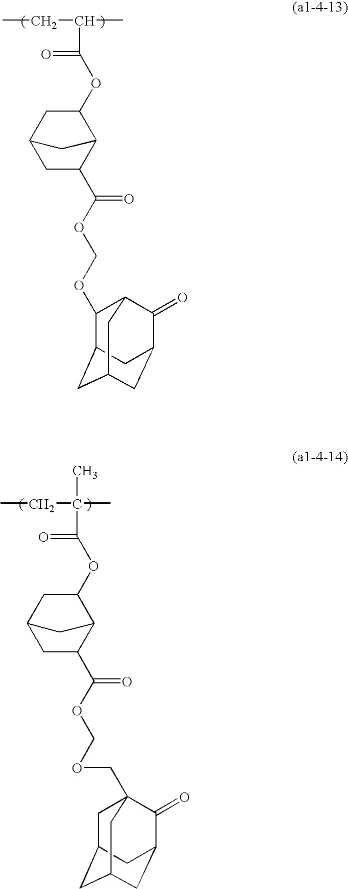

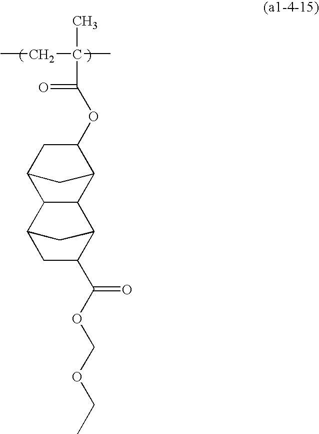

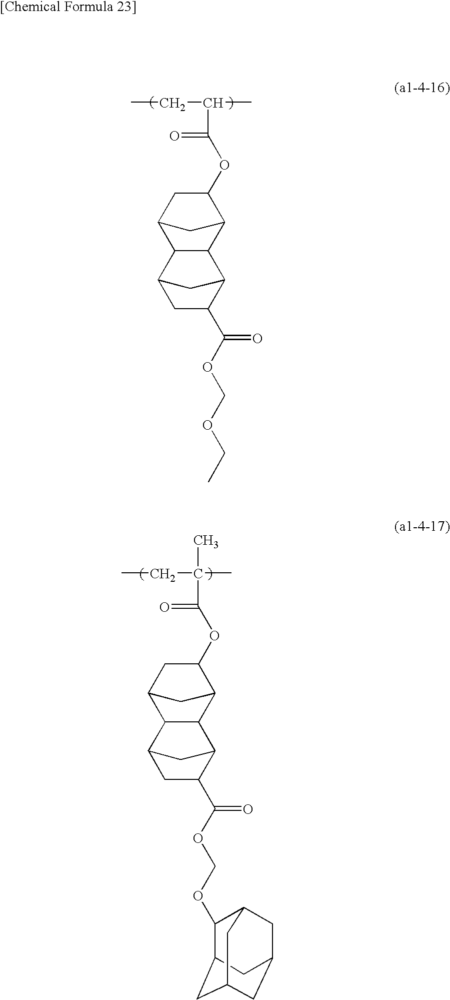

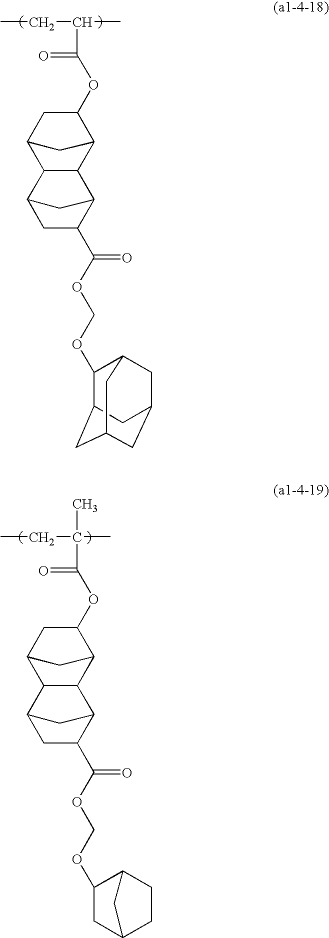

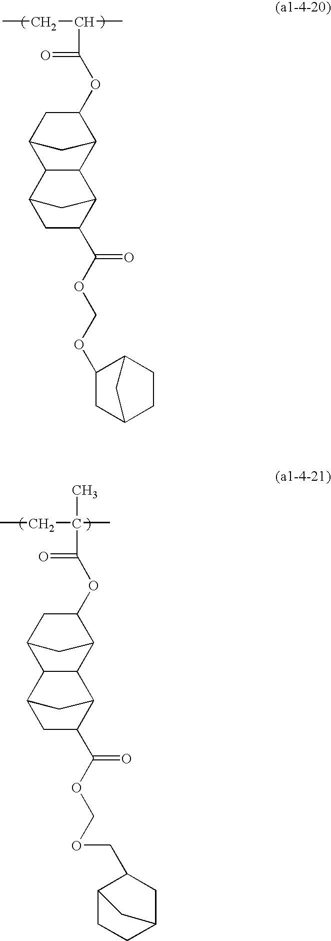

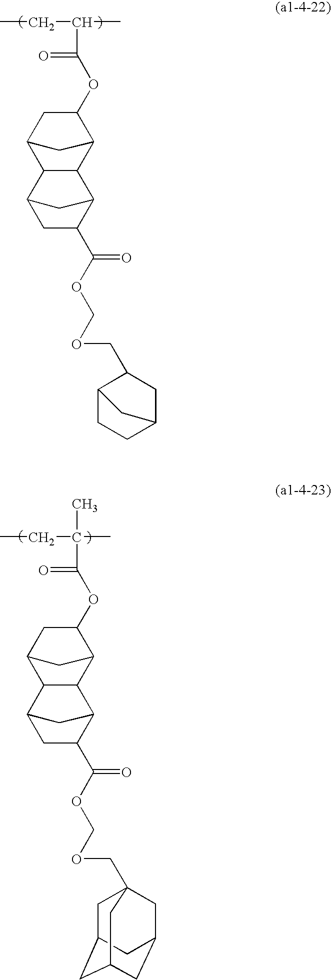

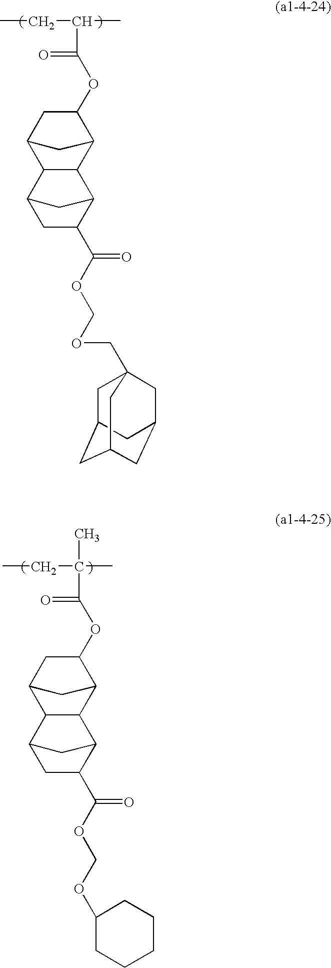

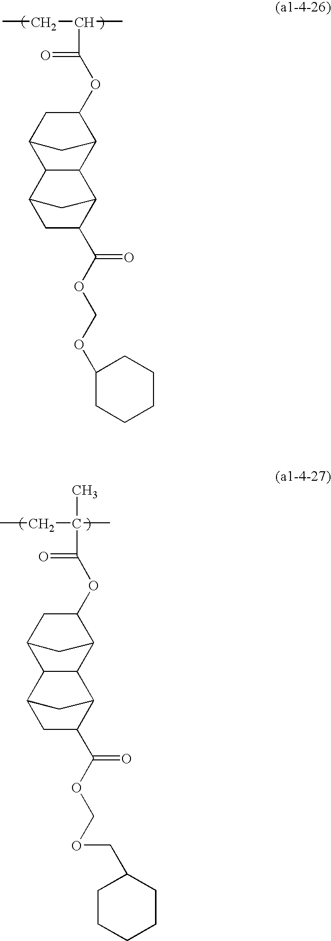

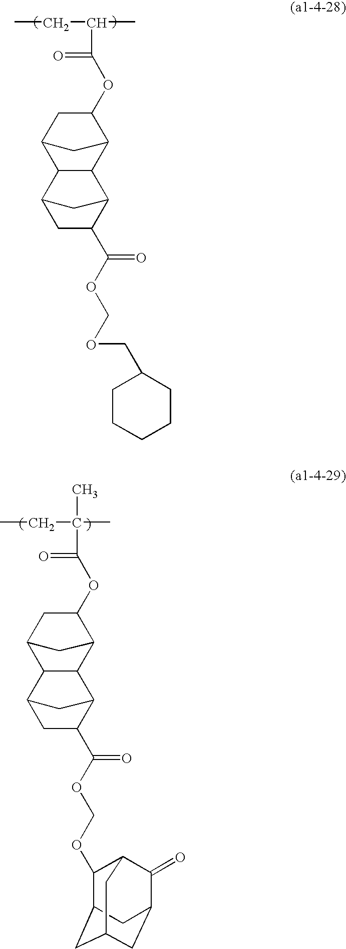

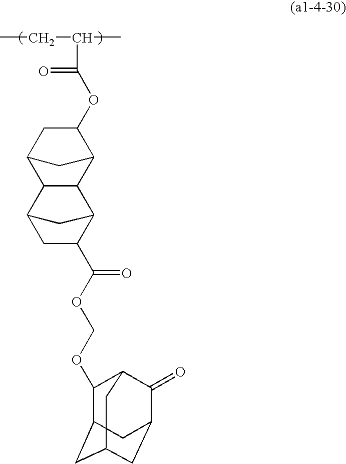

As the structural unit (a1), one type can be used alone, or two or more different types can be used in combination.

Among these, a structural unit represented by the general formula (a1-1) is preferable. More specifically, at least one structural unit selected from the group consisting of structural units represented by the formulae (a1-1-1) to (a1-1-6) or (a1-1-35) to (a1-1-41) can be preferably used.

Further, as the structural unit (a1), structural units represented by a general formula (a1-1-01) shown below which includes the structural units represented by formulae (a1-1-1) to (a1-1-4), and structural units represented by a general formula (a1-1-02) shown below which includes the structural units represented by formulae (a1-1-35) to (a1-1-41) are also preferable.

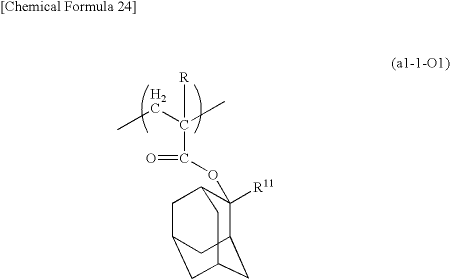

(In the formula, R represents a hydrogen atom, a halogen atom, a lower alkyl group or a halogenated lower alkyl group; and R¹¹ represents a lower alkyl group.)

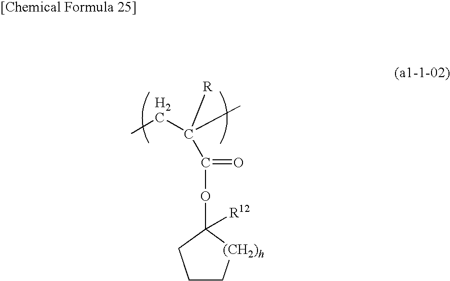

(In the formula, R represents a hydrogen atom, a halogen atom, a lower alkyl group or a halogenated lower alkyl group; R¹² represents a lower alkyl group; and h represents an integer of 1 to 3.)

In the general formula (a1-1-01), R is the same as R described in the aforementioned general formula (a1-1). The lower alkyl group for R¹¹ is the same as the lower alkyl group described above in R, and is preferably a methyl group or an ethyl group.

In the general formula (a1-1-02), R is the same as R described in the aforementioned general formula (a1-1). The lower alkyl group for R¹² is the same as the lower alkyl group described above in R. R¹² is preferably a methyl group or an ethyl group, and most preferably an ethyl group. h is preferably 1 or 2, and most preferably 2.

In the component (A1), the amount of the structural unit (a1) based on the combined total of all structural units constituting the component (A1) is preferably 10 to 80 mol %, more preferably 20 to 70 mol %, and still more preferably 25 to 50 mol %. When this proportion is not less than the lower limit in the above range, then a pattern can be easily formed using a positive resist composition which includes the component (a1), whereas when the proportion is not more than the upper limit in the above range, a good quantitative balance with the other components can be attained.

Structural Unit (a2)

In the present invention, the component (A1) preferably has a structural unit (a2) derived from an acrylate ester having a lactone-containing cyclic group, in addition to the structural unit (a1).

Here, the term “lactone-containing cyclic group” means a cyclic group containing a single ring (lactone ring) which has a “—O—C(O)—” structure. This lactone ring is counted as the first ring, and groups that contain only the lactone ring are referred to as monocyclic groups, whereas groups that also contain other ring structures are described as polycyclic groups regardless of the structure of the other rings.

In the case of using the component (A1) to form a resist film, the lactone-containing cyclic group of the structural unit (a2) is effective at improving the adhesion between the resist film and a substrate, and improving compatibility with the developing solution which contains water.

The structural unit (a2) can be used arbitrarily without any particular restriction.

Specific examples of the lactone-containing monocyclic group include a group in which one hydrogen atom is eliminated from γ-butyrolactone. Furthermore, specific examples of the lactone-containing polycyclic group include a group in which one hydrogen atom is eliminated from a bicycloalkane, a tricycloalkane, or a tetracycloalkane which contains a lactone ring.

Specific examples of the structural unit (a2) include structural units represented by the general formulae (a2-1) to (a2-5) shown below.

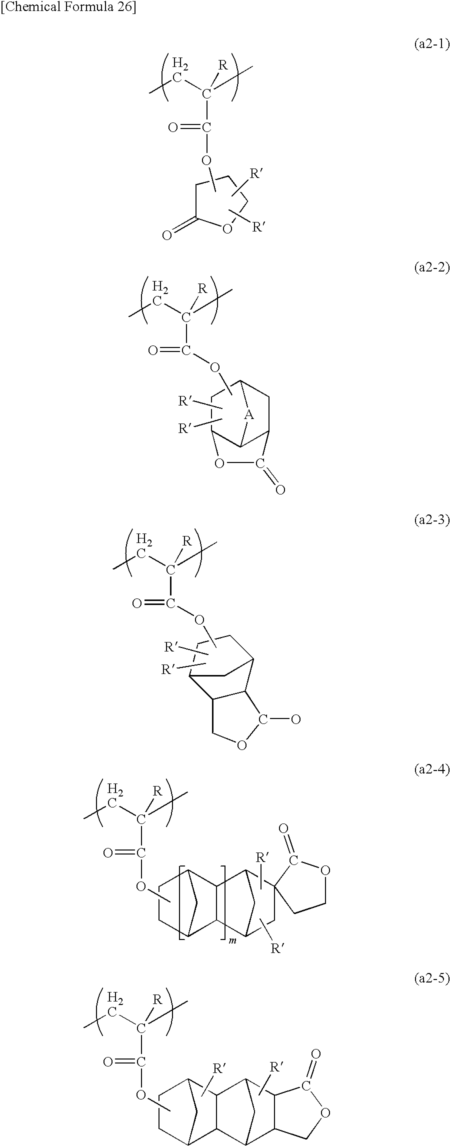

(In the formula, R represents a hydrogen atom, a halogen atom, a lower alkyl group or a halogenated lower alkyl group; R′ each independently represents a hydrogen atom, a lower alkyl group or an alkoxy group of 1 to 5 carbon atoms; m represents an integer of 0 or 1;

and A represents an alkylene group of 1 to 5 carbon atoms or an oxygen atom.)

R in the general formula (a2-1) to (a2-5) is the same as R described above in the structural unit (a1).

The lower alkyl group for R′ is the same as the lower alkyl group for R described above in the general formula (a1″) of the structural unit (a1).

Specific examples of alkylene groups of 1 to 5 carbon atoms for A include a methylene group, an ethylene group, an n-propylene group and an isopropylene group.

In the general formula (a2-1) to (a2-5), R′ is preferably a hydrogen atom in terms of industrial availability.

Specific examples of the structural units represented by the general formulae (a2-1) to (a2-5) include the following.

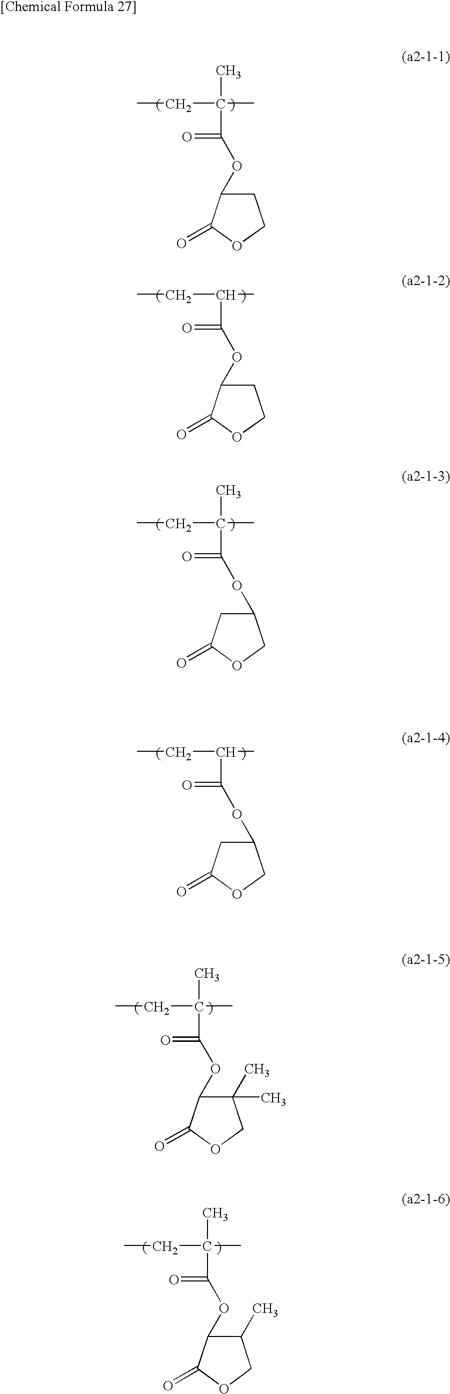

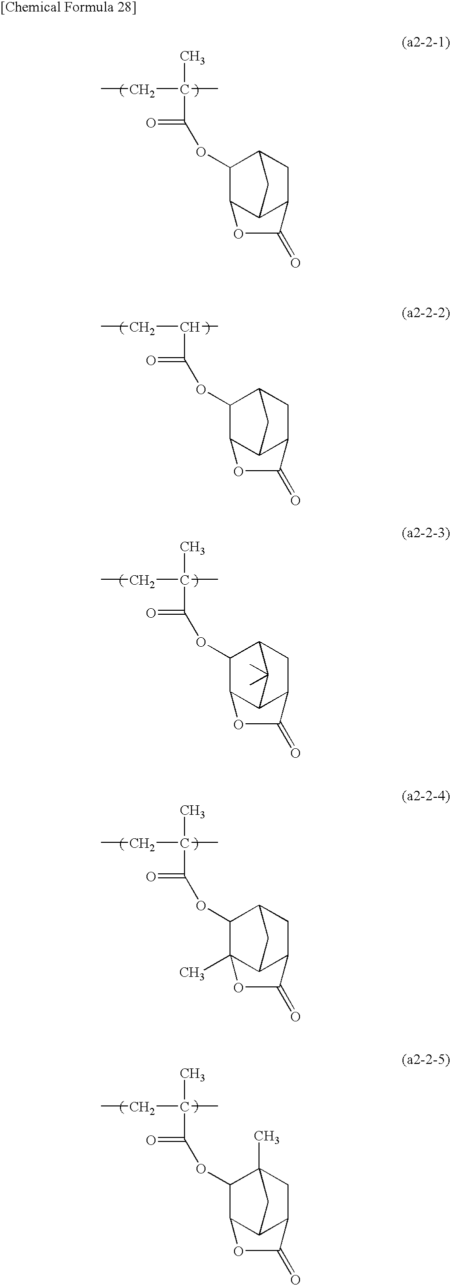

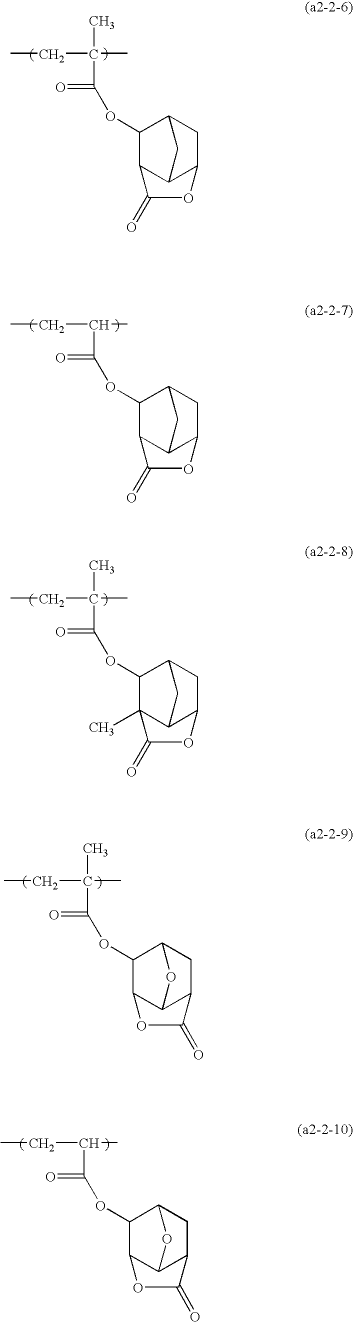

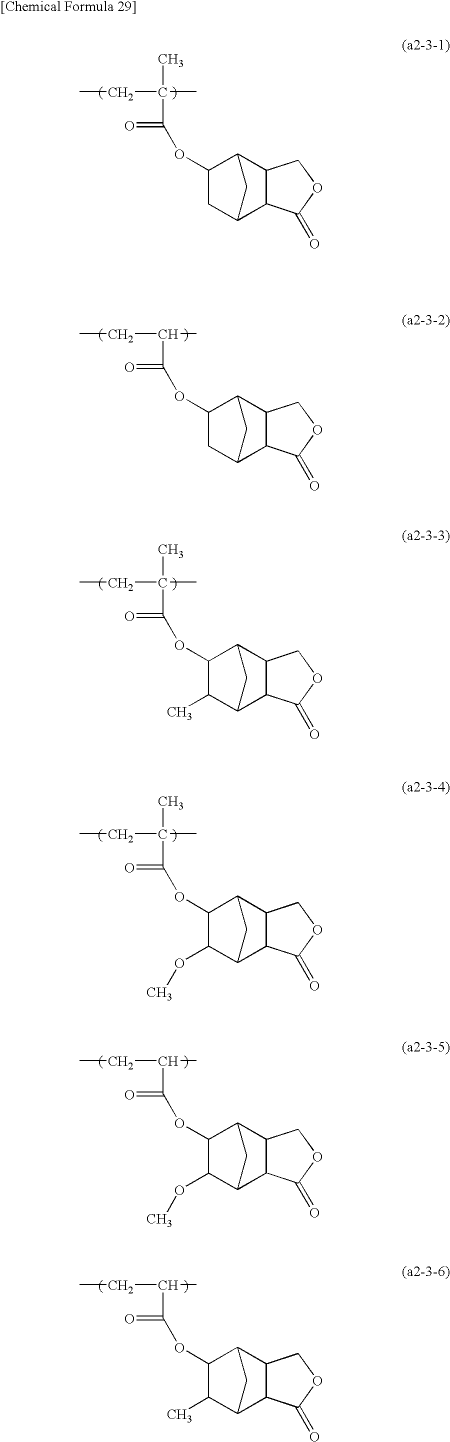

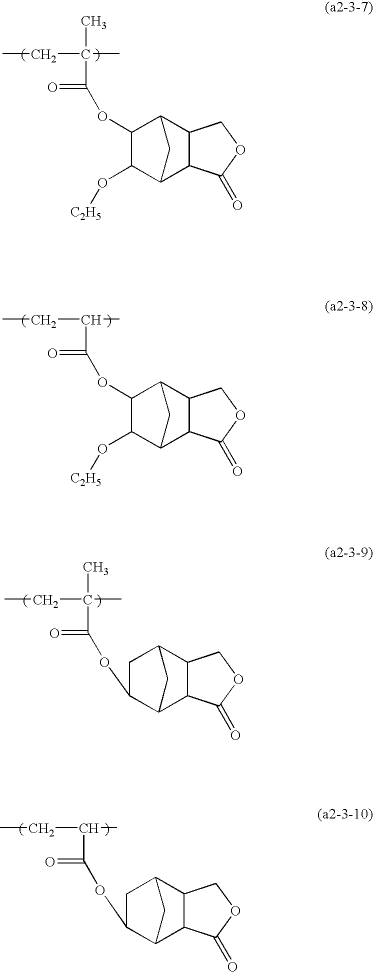

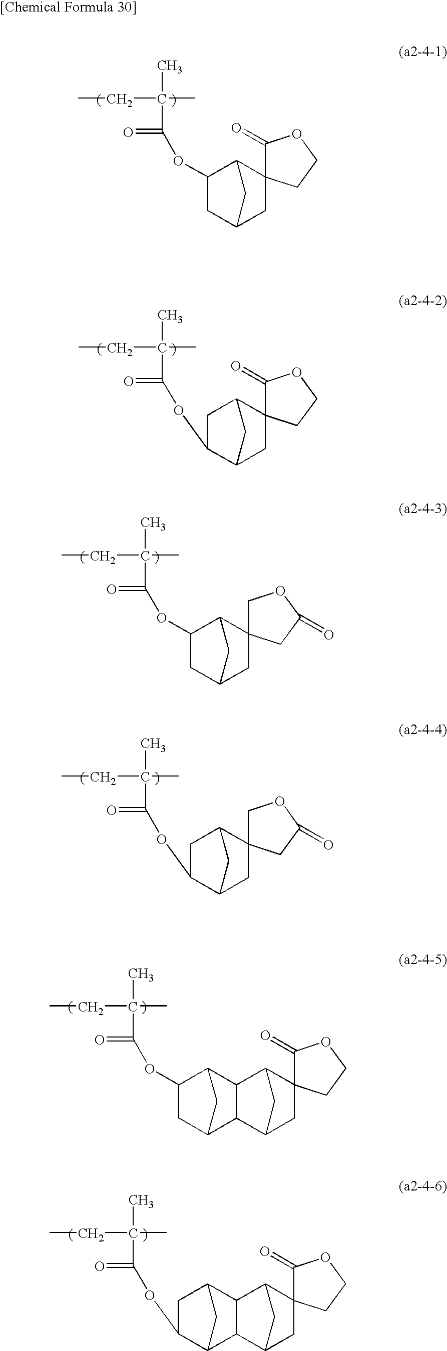

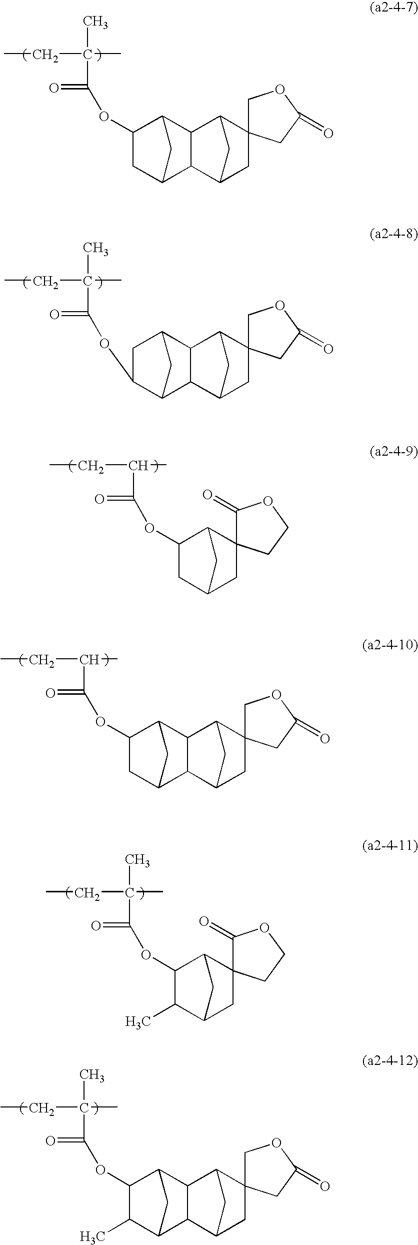

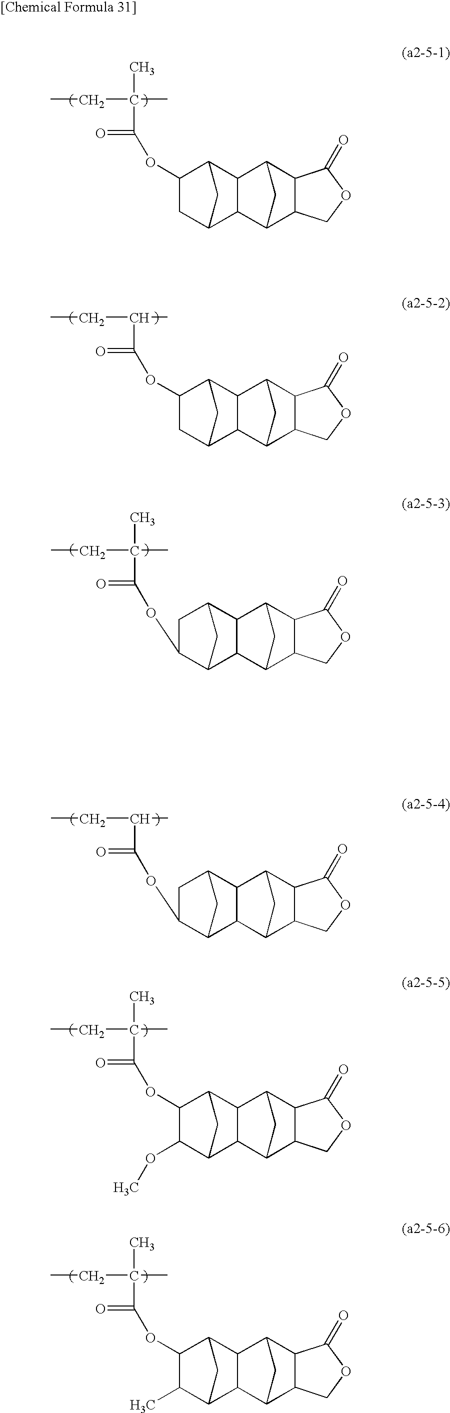

Of these, at least one structural unit selected from the group consisting of the structural units represented by the general formulae (a2-1) to (a2-5) is preferably used, and at least one structural unit selected from the group consisting of the structural units represented by the general formulae (a2-1) to (a2-3) is more preferably used. Specifically, at least one structural unit selected from the group consisting of the structural units represented by general formulae (a2-1-1), (a2-1-2), (a2-2-1), (a2-2-2), (a2-3-1), (a2-3-2), (a2-3-9) and (a2-3-10) is preferably used.

In the component (A1), as the structural unit (a2), one type may be used alone, or two or more types may be used in combination.

In the component (A1), the amount of the structural unit (a2) is preferably 5 to 60 mol %, more preferably 10 to 50 mol %, and still more preferably 20 to 50 mol %, based on the combined total of all structural units constituting the component (A1). When this proportion is not less than the lower limit in the above range, then the effect made by containing the structural unit (a2) can be sufficiently obtained. When the proportion is not more than the upper limit in the above range, a good quantitative balance with the other structural units can be attained.

Structural Unit (a3)

In the present invention, the component (A1) preferably has a structural unit (a3) derived from an acrylate ester having a polar group-containing aliphatic hydrocarbon group, in addition to the structural unit (a1) or the structural units (a1) and (a2). By including the structural unit (a3), hydrophilicity of the component (A1) is enhanced, thereby improving the compatibility with the developing solution, and improving the alkali solubility within the exposed portions of the resist. Therefore, the structural unit (a3) contributes to an improvement in resolution.

Examples of the polar group include a hydroxyl group, cyano group, carboxyl group, or hydroxyalkyl group (hereinafter, sometimes referred to as “fluorinated alkyl alcohol”) in which at least one of the hydrogen atoms of the alkyl group have been substituted with fluorine atoms, and of these, a hydroxyl group is particularly desirable.

Examples of the aliphatic hydrocarbon group include a linear or branched hydrocarbon group of 1 to 10 carbon atoms (preferably an alkylene group), and a polycyclic aliphatic hydrocarbon group (polycyclic group). The polycyclic group can be appropriately selected from the multitude of structural units proposed as resins in resist compositions for ArF excimer lasers and the like.

Of the various possibilities, structural units derived from an acrylate ester that include an aliphatic polycyclic group that contains a hydroxyl group, a cyano group, a carboxyl group or a hydroxyalkyl group in which at least one of the hydrogen atoms of the alkyl group have been substituted with fluorine atoms are more preferable. Examples of the polycyclic group include groups in which one or more hydrogen atoms have been removed from a polycycloalkane such as a bicycloalkane, a tricycloalkane, and a tetracycloalkane. Specific examples include a group in which one or more hydrogen isobornane, tricyclodecane, or tetracyclododecane. Of these polycyclic groups, a group in which two or more hydrogen atoms have been removed from adamantane, norbornane, or tetracyclododecane is industrially preferable.

As the structural unit (a3), for example, a structural unit derived from a hydroxyethyl ester of acrylic acid is preferable, when the hydrocarbon group within the polar group-containing aliphatic hydrocarbon group is a linear or branched hydrocarbon group of 1 to 10 carbon atoms. On the other hand, a structural unit represented by the general formula (a3-1), (a3-2), or (a3-3) shown below is preferable, when the hydrocarbon group is a polycyclic group.

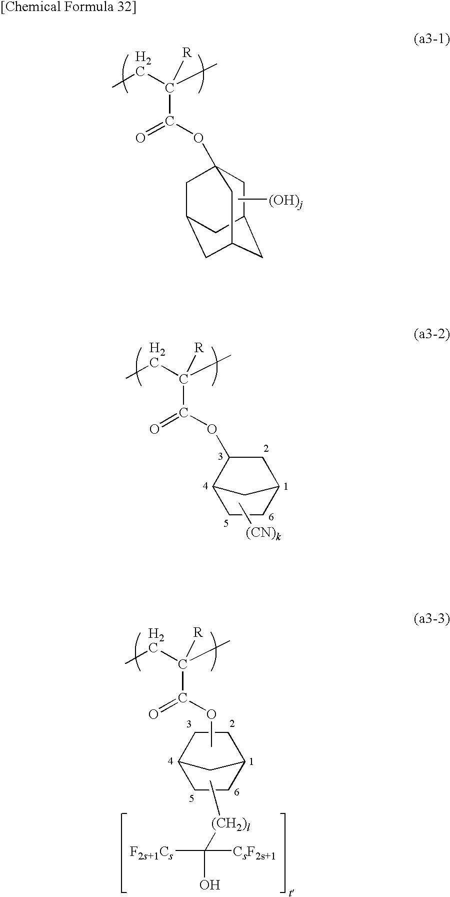

(In the formula, R represents a hydrogen atom, a halogen atom, a lower alkyl group, or a halogenated alkyl group; j represents an integer of 1 to 3; k represents an integer of 1 to 3; t′ represents an integer of 1 to 3; 1 represents an integer of 1 to 5; and s represents an integer of 1 to 3.)

In the general formulae (a3-1) to (a3-3), the halogen atom, lower alkyl group or halogenated lower alkyl group for R are the same as the halogen atom, lower alkyl group or halogenated lower alkyl group which can be bonded with the α-position of the aforementioned acrylate ester.

In the general formula (a3-1), j is preferably 1 or 2, and more preferably 1. In the case that j is 2, a structural unit in which a hydroxyl group is bonded with the 3-position and 5-position of the adamantyl group is preferable. In the case that j is 1, a structural unit in which a hydroxyl group is bonded with the 3-position of the adamantyl group is preferable.

Of these, it is preferable that j be 1, and the hydroxyl group be bonded with the 3-position of the adamantyl group.

In the general formula (a3-2), k is preferably 1. In the general formula (a3-2), a cyano group is preferably bonded with the 5-position or 6-position of the norbornyl group.

In the general formula (a3-3), t′ is preferably 1. 1 is preferably 1. s is preferably 1. Further, in the general formula (a3-3), it is preferable that a 2-norbonyl group or 3-norbonyl group be bonded at the terminal of the carboxy group of the acrylic acid. It is preferable that a fluorinated alkyl alcohol within brackets [ ] in the formula (a3-3) be bonded with the 5-position or 6-position of the norbornyl group.

In the component (A1), as the structural unit (a3), one type may be used alone, or two or more types may be used in combination.

In the component (A1), the amount of the structural unit (a3) is preferably 5 to 50 mol %, more preferably 5 to 40 mol %, and still more preferably 5 to 25 mol %, based on the combined total of all structural units constituting the component (A1). When this proportion is not less than the lower limit in the above range, then the effect made by containing the structural unit (a3) can be sufficiently obtained. When the proportion is not more than the upper limit in the above range, a good quantitative balance with the other structural units can be attained.

Structural Unit (a4)

The component (A1) may also have a structural unit (a4) which is different from the above-mentioned structural units (a1) to (a3), as long as the effects of the present invention are not impaired.

As the structural unit (a4), any other structural unit which cannot be classified as one of the above structural units (a1) to (a3) can be used without any particular limitations, and any of the multitude of conventional structural units used within resist resins for ArF excimer lasers or KrF excimer lasers (and particularly for ArF excimer lasers) can be used.

The structural unit (a4) is preferably, for example, a structural unit derived from an acrylate ester containing a non-acid-dissociable aliphatic polycyclic group. Examples of the polycyclic group include the same groups as those described above in the structural unit (a1), and any of the multitude of conventional polycyclic groups used within the resin component of resist compositions for ArF excimer lasers or KrF excimer lasers (and preferably for ArF excimer lasers) can be used.

In particular, at least one group selected from amongst a tricyclodecanyl group, an adamantyl group, a tetracyclododecanyl group, an isobornyl group, and a norbornyl group is preferable in terms of industrial availability and the like. These polycyclic groups may contain a linear or branched alkyl group of 1 to 5 carbon atoms as a substituent group.

Specific examples of the structural unit (a4) include a structural unit represented by the general formulae (a4-1) to (a4-5) shown below.

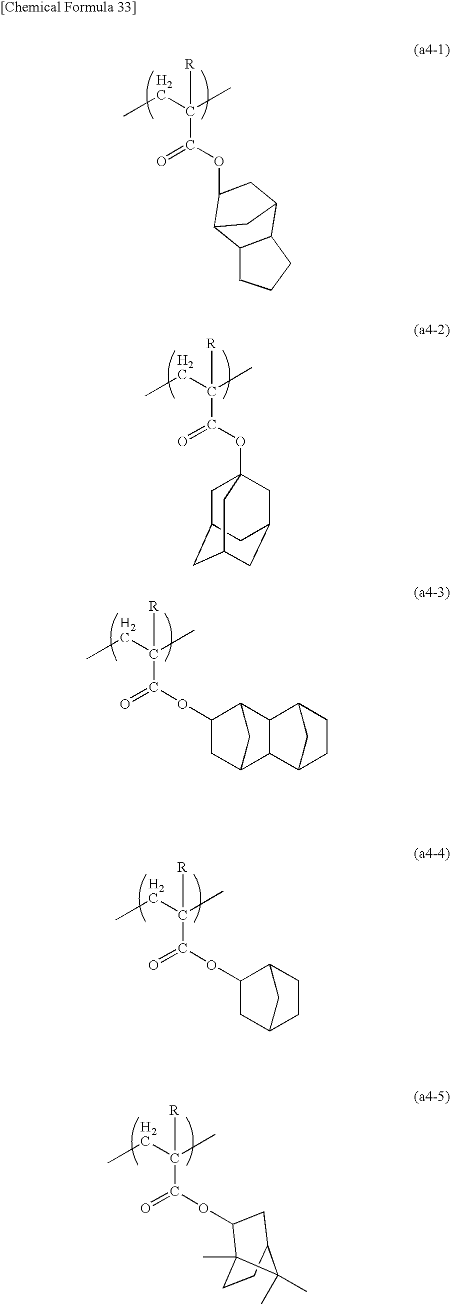

(In the formula, R represents a hydrogen atom, a halogen atom, a lower alkyl group, or a halogenated lower alkyl group.)

The halogen atom, lower alkyl group, and halogenated lower alkyl group for R in the general formulae (a4-1) to (a4-5) is the same as the halogen atom, lower alkyl group and halogenated alkyl group which may be bonded with the α-position of the acrylate ester.

When the structural unit (a4) is included in the component (A1), the amount of the structural unit (a4) based on the combined total of all the structural units that constitute the component (A1) is preferably within the range from 1 to 30 mol %, and more preferably from 10 to 20 mol %.

In the present invention, the component (A1) is a resin component (polymer) which exhibits increased solubility in an alkali developing solution under action of acid. As such a resin component (polymer), a copolymer having the structural units (a1), (a2) and (a3) can be preferably used. Examples of such a copolymer include a copolymer consisting of the structural units (a1), (a2) and (a3), and a copolymer consisting of the structural units (a1), (a2), (a3) and (a4).

In the present invention, as the component (A), a copolymer (A1-1) including a combination of structural units represented by a general formula (A1-1) shown below is particularly preferable.

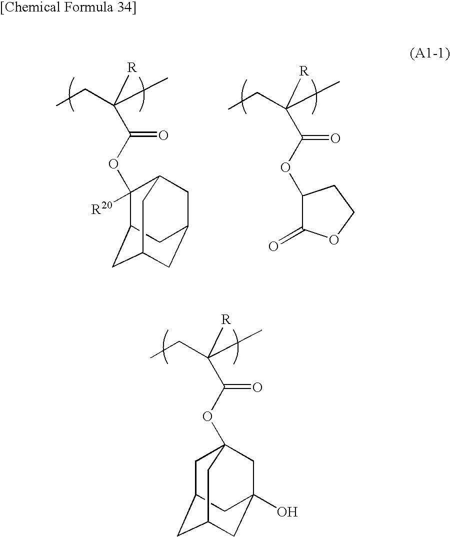

(In the formula, R represents a hydrogen atom, a halogen atom, a lower alkyl group or a halogenated alkyl group; and R²⁰ represents a lower alkyl group).

In the formula (A1-1), the halogen atom, lower alkyl group or halogenated lower alkyl group for R is the same as the halogen atom, lower alkyl group, or halogenated lower alkyl group which may be bonded with α-position of the above-mentioned acrylate ester. Of these, R is most preferably a hydrogen atom or a methyl group.

R²⁰ represents a lower alkyl group, is preferably a methyl group or an ethyl group, and most preferably a methyl group.

In the component (A), as the copolymer (A1-1), one kind can be used alone, or two or more kinds can be used in combination.

In the component (A), the content of the copolymer (A1-1) is preferably 70% by weight or more, more preferably 80% by weight or more, and most preferably 100% by weight. When the total content is not less than the lower limit of the above range, the lithography properties as a positive resist composition can be improved.

The component (A1) can be obtained, for example, by a conventional radical polymerization or the like of the monomers corresponding with each of the structural units, using a radical polymerization initiator such as azobisisobutyronitrile (AIBN).

Furthermore, in the component (A1), by using a chain transfer agent such as a HS—CH₂—CH₂—CH₂—C(CF₃)₂—OH, a —C(CF₃)₂—OH group can be introduced at the terminals of the component (A1). When a hydroxyalkyl group in which a part of the hydrogen atoms of the alkyl group has been substituted with fluorine atoms is introduced into a copolymer in this manner, the copolymer thus obtained can have an advantageous effect of reducing the levels of developing defects and LER (line edge roughness: non-uniform irregularities within the line side walls).

The weight average molecular weight (Mw) (the polystyrene equivalent value determined by gel permeation chromatography) of the component (A1) is not particularly limited, and is preferably 2,000 to 50,000, more preferably 3,000 to 30,000, and most preferably 5,000 to 20,000. By ensuring that the weight average molecular weight of the polymer compound (A1) is no more than the upper limit, solubility sufficient for a resist relative to a resist solvent can be obtained. By ensuring that it is no less than the lower limit, excellent dry-etching resistance and excellent cross-sectional shape of the resist pattern can be obtained.

Further, the dispersity (Mw/Mn) is preferably within a range of 1.0 to 5.0, more preferably 1.0 to 3.0, and most preferably 1.2 to 2.5. Herein, Mn represents the number average molecular weight.

Further, as the component (A1), an alkali-soluble resin component other than the copolymer (A1-1), such as other polymeric compounds used in conventional positive resist compositions, may be used.

In the positive resist composition of the present invention, the content of the component (A1) may be adjusted according to the thickness of the resist film to be formed.

### <Component (B)>

In the resist composition of the present invention, the component (B) includes an acid generator (B1) (hereinafter, referred to as component (B1)) composed of the compound represented by the above general formula (b1-2). In the formula, R⁴¹, R⁴², R⁴³, n₁, n₂, n₃, and X⁻ are the same as those described in the compound according to the first aspect of the present invention.

By including the component (B1) in the component (B), solubility of the component (B) becomes satisfactory in a commonly-used resist solvent such as propylene glycol monomethyl ether (PGME), propylene glycol monomethyl ether acetate (PGMEA), and ethyl lactate (EL). Further, in a method of forming a resist pattern including immersion exposure or in a method of forming a resist pattern including formation of a triple-layer resist laminate, the use of the component (B1) in a resist composition for immersion lithography or a resist composition for forming an upper-layer resist film enables excellent lithography properties to be obtained.

Furthermore, the component (B1) can be blended much amount in a resist composition used for a method of forming a resist pattern including immersion exposure or a method of forming a resist pattern including formation of a triple-layer resist laminate. It is considered that this is attributed to high transparency (effective suppression of photoabsorption) in the exposure wavelength range (especially, wavelength range for ArF excimer laser).

As the component (B), one type can be used alone, or two or more types can be used in combination.

In the resist composition of the present invention, the content of the component (B1) in the entire component (B) is preferably 40% by weight or more, more preferably 70% by weight or more, and most preferably 100% by weight. When the content is not less than the lower limit of the above range, the resist pattern shape is excellent. In particular, when a resist pattern is formed using a resist composition for immersion lithography or a resist composition for forming an upper-layer resist film, the resist composition including the component (B1) whose content is not less than the lower limit enables lithography properties to be improved. When a triple-layer resist laminate is formed, the resist composition including the component (B1) whose content is not less than the lower limit is advantageous in that the compatibility of the resist with the lower-layer film, and enables footing of the resist pattern and the like to be suppressed, Therefore, the component (B1) whose content is not less than the lower limit is preferable.

Further, in the resist composition of the present invention, the amount of the component (B1) is preferably 1 to 30 parts by weight, more preferably 5 to 20 parts by weight, and most preferably 7 to 18 parts by weight, relative to 100 parts by weight of the component (A). The component (B1) whose content is not less than the lower limit of the above-mentioned range enables the lithography properties to be improved, when a resist pattern is formed using a resist composition for immersion lithography or a resist composition for forming an upper-layer resist film which includes the component (B1). On the other hand, when the content is not more than the upper limit, storage stability can be excellent.

In the component (B), an acid generator (B2) (hereinafter, referred to as component (B2)) other than the component (B1) may be used in combination with the component (B1).

There are no particular restrictions on the component (B2) as long as it is a component other then the component (B1), and those proposed as acid generators for chemically-amplified resists can be used as the component (B2).

Examples of these acid generators are numerous, and include onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzylsulfonate-based acid generators; iminosulfonate-based acid generators; and disulfone-based acid generators.

As an onium salt-based acid generator, for example, an acid generator represented by the general formula (b-0) shown below can be preferably used.

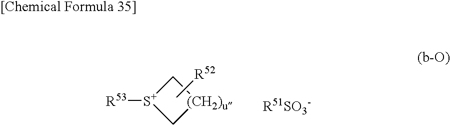

(In the formula, R⁵¹ represents a linear, branched, or cyclic alkyl group, or a linear, branched, or cyclic fluorinated alkyl group; R⁵² represents a hydrogen atom, a hydroxyl group, a halogen atom, a linear or branched alkyl group, a linear or branched halogenated alkyl group, or a linear or branched alkoxy group; R represents an aryl group which may contain a substituent group; and u″ represents an integer of 1 to 3.)

In the general formula (b-0), R⁵¹ represents a linear, branched or cyclic alkyl group, or a linear, branched or cyclic fluorinated alkyl group.

The number of carbon atoms in the linear or branched alkyl group for R⁵¹ is preferably 1 to 10, more preferably 1 to 8, and most preferably 1 to 4.

The number of carbon atoms in the cyclic alkyl group for R⁵¹ is preferably 4 to 12, more preferably 5 to 10, and most preferably 6 to 10.

The number of carbon atoms in the linear or branched fluorinated alkyl group for R⁵¹ is preferably 1 to 10, more preferably 1 to 8, and most preferably 1 to 4.

The number of carbon atoms in the cyclic fluorinated alkyl group for R⁵¹ is preferably 4 to 12, more preferably 5 to 10, and most preferably 6 to 10.

Also, the fluorination rate of the fluorinated alkyl group (proportion of substituted fluorine atoms relative to all hydrogen atoms before substitution in the alkyl group) is preferably within a range of 10 to 100%, more preferably 50 to 100%, and particularly preferably those wherein all hydrogen atoms are substituted with fluorine atoms, because the strength of the acid increases.

R⁵¹ is most preferably a linear alkyl group or a linear fluorinated alkyl group.

R⁵² represents a hydrogen atom, a hydroxyl group, a halogen atom, a linear or branched alkyl group, a linear or branched halogenated alkyl group, or a linear or branched alkoxy group.

Examples of the halogen atom for R⁵² include a fluorine atom, a bromine atom, a chlorine atom and an iodine atom. Of these, a fluorine atom is preferable.

The alkyl group for R⁵² is linear or branched, and the number of carbon atoms in the alkyl group is preferably 1 to 5, more preferably 1 to 4, and most preferably 1 to 3.

The halogenated alkyl group for R⁵² is a group in which a part of or all of hydrogen atoms in the alkyl group are substituted with halogen atoms. As the alkyl group in the halogenated alkyl group, the same alkyl group as “alkyl group” for R⁵² can be used. As the halogen atom with which the hydrogen atom is substituted, the same halogen atoms as those described above in “halogen atoms” can be used. In the halogenated alkyl group, 50 to 100% of all the hydrogen atoms are preferably substituted with halogen atoms, and it is more preferable that all of the hydrogen atoms are substituted with halogen atoms.

The alkoxy group for R⁵² is linear or branched, and the number of carbon atoms in the alkoxy group is preferably 1 to 5, more preferably 1 to 4, and most preferably 1 to 3.

Of these, R⁵² is preferably a hydrogen atom.

R⁵³ represents an aryl group which may have a substituent group. Examples of the basic ring from which a substituent group is removed include a naphthyl group, a phenyl group and an anthracenyl group. Of these, a phenyl group is preferable in terms of the effects of the present invention and the excellent absorption relative to exposure light such as ArF excimer lasers.

Examples of the substituent group include a hydroxyl group and a lower alkyl group (linear or branched, and preferably has 1 to 5 carbon atoms, and is more preferably a methyl group).

The aryl group for R⁵³ is more preferably that which has no substituent group. u″ represents an integer of 1 to 3, preferably 2 or 3, and more preferably 3.

Suitable examples of the acid generator represented by the general formula (b-0) include the following.

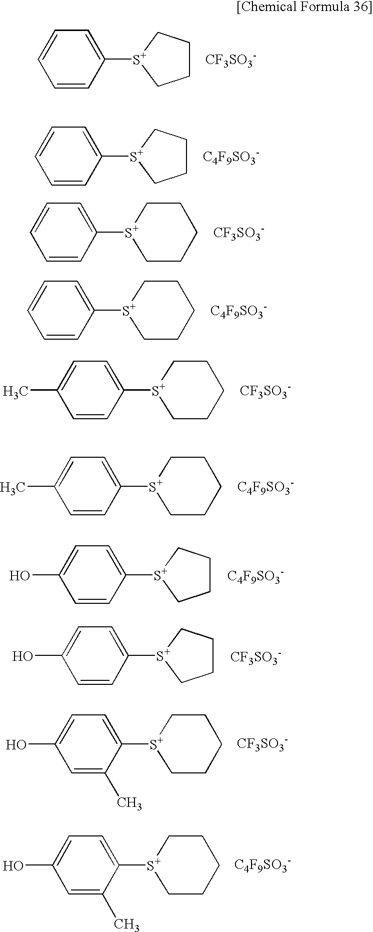

As the acid generator represented by the general formula (b-0), one type may be used alone, or two or more types may be used in combination.

Also, as an onium salt-based acid generator other than the acid generator represented by the general formula (b-0), a compound represented by a general formula (b-1) or (b-2) shown below can be preferably used.

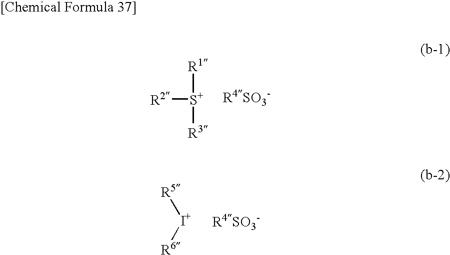

(In the formula, R¹″ to R³″, R⁵″ and R⁶″ each independently represents an aryl group or an alkyl group; R⁴″ represents a linear, branched or cyclic alkyl group, or a linear, branched or cyclic fluorinated alkyl group; at least one of R¹″ to R³″ represents an aryl group; and at least one of R⁵″ and R⁶″ represents an aryl group.)

In the general formula (b-1), R¹″ to R³″ each independently represents an aryl group or an alkyl group. At least one of R¹″ to R³″ represents an aryl group. Two or more of R¹″ to R³″ are preferably aryl groups, and all of R¹″ to R³″ are most preferably aryl groups.

There is no particular restriction on the aryl group for R¹″ to R³″. For example, the aryl group may be an aryl group of 6 to 20 carbon atoms, and a part of or all of hydrogen atoms in the aryl group may be substituted with an alkyl group, an alkoxy group, a halogen atom and the like, or may be not substituted. The aryl group is preferably an aryl group of 6 to 10 carbon atoms because it can be synthesized inexpensively. Specific examples thereof include a phenyl group and a naphthyl group.

In the aryl group for R¹″ to R³″, the alkyl group with which hydrogen atoms may be substituted is preferably an alkyl group of 1 to 5 carbon atoms, and most preferably a methyl group, an ethyl group, a propyl group, an n-butyl group, and a tert-butyl group.

In the aryl group for R¹″ to R³″, the alkoxy group with which hydrogen atoms may be substituted is preferably an alkoxy group of 1 to 5 carbon atoms, and most preferably a methoxy group or an ethoxy group.

In the aryl group for R¹″ to R³″, the halogen atom with which hydrogen atoms may be substituted is preferably a fluorine atom.

There is no restriction on the alkyl groups for R¹″ to R³″. Examples thereof include a linear, branched, or cyclic alkyl group of 1 to 10 carbon atoms. Specific examples include a methyl group, an ethyl group, an n-propyl group, an isopropyl group, an n-butyl group, an isobutyl group, an n-pentyl group, a cyclopentyl group, a hexyl group, a cyclohexyl group, a nonyl group, and a decanyl group. Of these, the alkyl group preferably has 1 to 5 carbon atoms because it is excellent in resolution, and a methyl group is most preferable because it is excellent in resolution and can be synthesized inexpensively.

Of these, it is most preferable that R¹″ to R³″ each be independently a phenyl group or a naphthyl group.

R⁴″ represents a linear, branched or cyclic alkyl group, or a linear, branched or cyclic fluorinated alkyl group.

The number of carbon atoms in the linear or branched alkyl group for R⁴″ is preferably 1 to 10, more preferably 1 to 8, and most preferably 1 to 4.

The cyclic alkyl group for R⁴″ is the same as the cyclic group described above in R¹″. The number of carbon atoms in the cyclic alkyl group of R⁴″ is preferably 4 to 15, more preferably 4 to 10, and most preferably 6 to 10.

The number of carbon atoms in the linear or branched fluorinated alkyl group for R⁴″ is preferably 1 to 10, more preferably 1 to 8, and most preferably 1 to 4.

The number of carbon atoms in the cyclic fluorinated alkyl group for R⁴″ is preferably 4 to 15, more preferably 4 to 10, and most preferably 6 to 10.

Furthermore, the fluorination rate of the fluorinated alkyl group (proportion of fluorine atoms in the alkyl group) is preferably within the range of 10 to 100%, more preferably 50 to 100%, and particularly preferably those wherein all hydrogen atoms are substituted with fluorine atoms, because the strength of the acid increases.

R⁴″ is most preferably a linear or cyclic alkyl group, or a linear or cyclic fluorinated alkyl group.

In the general formula (b-2), R⁵″ and R⁶″ each independently represents an aryl group or an alkyl group. At least one of R⁵″ and R⁶″ represents an aryl group. It is preferable that both R⁵″ and R⁶″ represent an aryl group.

As the aryl group for R⁵″ and R⁶″, the same aryl groups as those for R¹″ to R³″ can be used.

As the alkyl group for R⁵″ and R⁶″, the same alkyl groups as those for R¹″ to R³″ can be used.

It is most preferable that both R⁵″ and R⁶″ represent a phenyl group.

As R⁴″ in the general formula (b-2), the same groups as those described in R⁴″ in the general formula (b-1) can be used.

Specific examples of suitable onium salt-based acid generators represented by formula (b-1) or (b-2) include diphenyliodonium trifluoromethanesulfonate or nonafluorobutanesulfonate; bis(4-tert-butylphenyl)iodonium trifluoromethanesulfonate or nonafluorobutanesulfonate; triphenylsulfonium trifluoromethanesulfonate, heptafluoropropanesulfonate or nonafluorobutanesulfonate; tri(4-methylphenyl)sulfonium trifluoromethanesulfonate, heptafluoropropanesulfonate or nonafluorobutanesulfonate; dimethyl(4-hydroxynaphthyl)sulfonium trifluoromethanesulfonate, heptafluoropropanesulfonate or nonafluorobutanesulfonate; monophenyldimethylsulfonium trifluoromethanesulfonate, heptafluoropropanesulfonate or nonafluorobutanesulfonate; diphenylmonomethylsulfonium trifluoromethanesulfonate, heptafluoropropanesulfonate or nonafluorobutanesulfonate; (4-methylphenyl)diphenylsulfonium trifluoromethanesulfonate, heptafluoropropanesulfonate or nonafluorobutanesulfonate; (4-methoxyphenyl)diphenylsulfonium trifluoromethanesulfonate, heptafluoropropanesulfonate or nonafluorobutanesulfonate; tri(4-tert-butyl)phenylsulfonium trifluoromethanesulfonate, heptafluoropropanesulfonate or nonafluorobutanesulfonate; diphenyl(1-(4-methoxy)naphthyl)sulfonium trifluoromethanesulfonate, heptafluoropropanesulfonate or nonafluorobutanesulfonate; and di(1-naphthyl)phenylsulfonium trifluoromethanesulfonate, heptafluoropropanesulfonate or nonafluorobutanesulfonate. Also, onium salts whose anion moiety is substituted with a methansulfonate, an n-propanesulfonate, an n-butanesulfonate, or an n-octanesulfonate can be used.

Further, an onium salt-based acid generator in which the anion moiety in the general formula (b-1) or (b-2) is substituted with an anion moiety represented by the general formula (b-3) or (b-4) shown below can also be used. Here, the cation moiety is the same as those described in the general formula (b-1) or (b-2).

In the present specification, the term “oxime sulfonate-based acid generator” means a compound which has at least one of the groups represented by the general formula (B-1) shown below, and has a property that generates an acid upon exposure to radiation. These kinds of oxime sulfonate-based acid generators are widely used for a chemically-amplified resist composition, so any oxime sulfonate-based acid generator, arbitrarily selected from these, can be used.

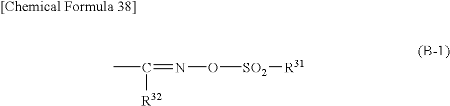

(In the formula (B-1), R³¹ and R³² each independently represents an organic group.)

The organic group for R³¹ or R³² is a group containing carbon atoms, and may further contain atoms other than carbon atoms (for example, a hydrogen atom, an oxygen atom, a nitrogen atom, a sulfur atom and a halogen atom (a fluorine atom, a chlorine atom and the like)).

The organic group for R³¹ is preferably a linear, branched or cyclic alkyl group or an aryl group. The alkyl group or aryl group may contain a substituent group. There is no particular restriction on the substituent group, and examples thereof include a fluorine atom, and a linear, branched or cyclic alkyl group of 1 to 6 carbon atoms. Here, the expression “having a substituent” means that at least one of or all of the hydrogen atoms in the alkyl group or the aryl group as the organic group for R³¹ are substituted with substituent groups.

The alkyl group as the organic group for R³¹ preferably has 1 to 20 carbon atoms, more preferably 1 to 10 carbon atoms, still more preferably 1 to 8 carbon atoms, still more preferably 1 to 6 carbon atoms, and most preferably 1 to 4 carbon atoms. Of these, as the alkyl group as the organic group for R³¹, a partially or completely halogenated alkyl group is particularly desirable. Here, the partially halogenated alkyl group means an alkyl group in which a part of the hydrogen atoms is substituted with halogen atoms, and the completely halogenated alkyl group means an alkyl group in which all of the hydrogen atoms are substituted with halogen atoms. Examples of the halogen atoms include a fluorine atom, a chlorine atom, a bromine atom and an iodine atom. Of these, a fluorine atom is preferable. That is, the halogenated alkyl group is preferably a fluorinated alkyl group.

The aryl group as the organic group for R³¹ preferably has 4 to 20 carbon atoms, more preferably 4 to 10 carbon atoms, and most preferably 6 to 10 carbon atoms Of these, as the aryl group as the organic group for R³¹, a partially or completely halogenated alkyl group is particularly desirable. Here, the partially halogenated aryl group means an aryl group in which a part of the hydrogen atoms is substituted with halogen atoms, and the completely halogenated aryl group means an aryl group in which all of the hydrogen atoms are substituted with halogen atoms. Examples of the halogen atoms include a fluorine atom, a chlorine atom, a bromine atom and an iodine atom. Of these, a fluorine atom is preferable.

R³¹ is particularly preferably an alkyl group of 1 to 4 carbon atoms containing no substituent group, or a fluorinated alkyl group of 1 to 4 carbon atoms.

The organic group for R³² is preferably a linear, branched or cyclic alkyl group, an aryl group, or a cyano group. As the alkyl group or the aryl group for R³², the same alkyl groups or aryl groups as those described above for R³¹ can be used.

R³² is particularly preferably a cyano group, an alkyl group of 1 to 8 carbon atoms containing no substituent group, or a fluorinated alkyl group of 1 to 8 carbon atoms.

Preferred examples of the oxime sulfonate-based acid generator include compounds represented by the general formula (B-2) or (B-3) shown below.

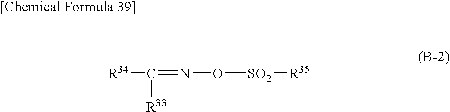

(In the general formula (B-2), R³³ represents a cyano group, an alkyl group containing no substituent group, or a halogenated alkyl group; R³⁴ represents an aryl group; and R³⁵ represents an alkyl group containing no substituent group, or a halogenated alkyl group.)

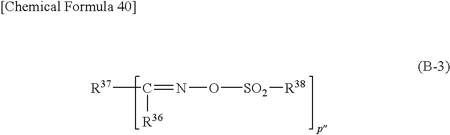

(In the general formula (B-3), R³⁶ represents a cyano group, an alkyl group containing no substituent group, or a halogenated alkyl group; R³⁷ represents a bivalent or trivalent aromatic hydrocarbon group; R³⁸ represents an alkyl group containing no substituent group, or a halogenated alkyl group; and p″ represents an integer of 2 or 3.)

In the general formula (B-2), the number of carbon atoms in the alkyl group containing no substituent group or the halogenated alkyl group for R³³ is preferably 1 to 10, more preferably 1 to 8, and most preferably 1 to 6. Examples of the halogen atoms in the halogenated alkyl group include a fluorine atom, a chlorine atom, a bromine atom and an iodine atom.

R³³ is preferably a halogenated alkyl group, and more preferably a fluorinated alkyl group.

The fluorinated alkyl group for R³³ is preferably a group in which 50% or more of the hydrogen atoms in the alkyl group are fluorinated, more preferably a group in which 70% or more of the hydrogen atoms in the alkyl group are fluorinated, and still more preferably a group in which 90% or more of the hydrogen atoms in the alkyl group are fluorinated.

Examples of the aryl group represented by R³⁴ include groups in which one hydrogen atom has been removed from an aromatic hydrocarbon ring, such as a phenyl group, a biphenyl group, a fluorenyl group, a naphthyl group, an anthracyl group, and a phenanthryl group; and heteroaryl groups in which a part of the carbon atoms which constitutes the ring(s) of these groups are substituted with heteroatoms such as an oxygen atom, a sulfur atom, and a nitrogen atom. Of these, a fluorenyl group is preferable.

The aryl group for R³⁴ may contain a substituent group such as an alkyl group of 1 to 10 carbon atoms, a halogenated alkyl group of 1 to 10 carbon atoms, and an alkoxy group of 1 to 10 carbon atoms. Examples of the halogen atoms in the halogenated alkyl group include a fluorine atom, a chlorine atom, a bromine atom and an iodine atom. The number of carbon atoms in the alkyl group or halogenated alkyl group for the substituent group is preferably 1 to 8, and more preferably 1 to 4. Also, the halogenated alkyl group is preferably a fluorinated alkyl group.

The number of carbon atoms in the alkyl group containing no substituent group or the halogenated alkyl group for R³⁵ is preferably 1 to 10, more preferably 1 to 8, and most preferably 1 to 6. Examples of the halogen atoms in the halogenated alkyl group include a fluorine atom, a chlorine atom, a bromine atom and an iodine atom.

R³⁵ is preferably a halogenated alkyl group, and more preferably a fluorinated alkyl group.

The fluorinated alkyl group for R³⁵ is preferably a group in which 50% or more of the hydrogen atoms in the alkyl group are fluorinated, more preferably a group in which 70% or more of the hydrogen atoms in the alkyl group are fluorinated, and still more preferably a group in which 90% or more of the hydrogen atoms in the alkyl group are fluorinated, because the strength of the generated acid increases. The fluorinated alkyl group for R³⁵ is most preferably a completely fluorinated alkyl group in which 100% of the hydrogen atoms are substituted with fluorine atoms.

In the general formula (B-3), as the alkyl group containing no substituent group or the halogenated alkyl group for R³⁶, the same alkyl groups containing no substituent group or halogenated alkyl groups as those described above in R³³ can be used.

Examples of the bivalent or trivalent aromatic hydrocarbon group for R³⁷ include aryl groups of R³⁴ in which one or two hydrogen atoms are further removed.

As the alkyl group containing no substituent group or the halogenated alkyl group for R³⁸, the same alkyl groups containing no substituent group or halogenated alkyl groups as those described above in R³⁵ can be used. p″ is preferably 2.

Specific examples of the oxime sulfonate-based acid generator include α-(p-toluenesulfonyloxyimino)-benzyl cyanide, α-(p-chlorobenzenesulfonyloxyimino)-benzylcyanide, α-(4-nitrobenzenesulfonyloxyimino)-benzylcyanide, α-(4-nitro-2-trifluoromethylbenzenesulfonyloxyimino)-benzylcyanide, α-(benzenesulfonyloxyimino)-4-chlorobenzylcyanide, α-(benzenesulfonyloxyimino)-2,4-dichlorobenzylcyanide, α-(benzenesulfonyloxyimino)-2,6-dichlorobenzylcyanide, α-(benzenesulfonyloxyimino)-4-methoxybenzylcyanide, α-(2-chlorobenzenesulfonyloxyimino)-4-methoxybenzylcyanide, α-(benzenesulfonyloxyimino)-thien-2-ylacetonitrile, α-(4-dodecylbenzenesulfonyloxyimino)-benzylcyanide, α-[(p-toluenesulfonyloxyimino)-4-methoxyphenyl]acetonitrile, α-[(dodecylbenzenesulfonyloxyimino)-4-methoxyphenyl]acetonitrile, α-(tosyloxyimino)-4-thienylcyanide, α-(methylsulfonyloxyimino)-1-cyclopentenylacetonitrile, α-(methylsulfonyloxyimino)-1-cyclohexenylacetonitrile, α-(methylsulfonyloxyimino)-1-cycloheptenylacetonitrile, α-(methylsulfonyloxyimino)-1-cyclooctenylacetonitrile, α-(trifluoromethylsulfonyloxyimino)-1-cyclopentenylacetonitrile, α-(trifluoromethylsulfonyloxyimino)-cyclohexylacetonitrile, α-(ethylsulfonyloxyimino)-ethylacetonitrile, α-(propylsulfonyloxyimino)-propylacetonitrile, α-(cyclohexylsulfonyloxyimino)-cyclopentylacetonitrile, α-(cyclohexylsulfonyloxyimino)-cyclohexylacetonitrile, α-(cyclohexylsulfonyloxyimino)-1-cyclopentenylacetonitrile, α-(ethylsulfonyloxyimino)-1-cyclopentenylacetonitrile, α-(isopropylsulfonyloxyimino)-1-cyclopentenylacetonitrile, α-(n-butylsulfonyloxyimino)-1-cyclopentenylacetonitrile, α-(ethylsulfonyloxyimino)-1-cyclohexenylacetonitrile, α-(isopropylsulfonyloxyimino)-1-cyclohexenylacetonitrile, α-(n-butylsulfonyloxyimino)-1-cyclohexenylacetonitrile, α-(methylsulfonyloxyimino)-phenylacetonitrile, α-(methylsulfonyloxyimino)-p-methoxyphenylacetonitrile, α-(trifluoromethylsulfonyloxyimino)-phenylacetonitrile, α-(trifluoromethylsulfonyloxyimino)-p-methoxyphenylacetonitrile, α-(ethylsulfonyloxyimino)-p-methoxyphenylacetonitrile, α-(propylsulfonyloxyimino)-p-methylphenylacetonitrile, and α-(methylsulfonyloxyimino)-p-bromophenylacetonitrile.

Also, oxime sulfonate-based acid generators disclosed in Japanese Unexamined Patent Application, First Publication No. Hei9-208554 ([Formula 18] and [Formula 19] in paragraphs [0012] to [0014]), and International Publication WO 2004/074242A2 (Examples 1 to 40 on pages 65 to 85) can be preferably used.

Further, suitable examples thereof include the following.

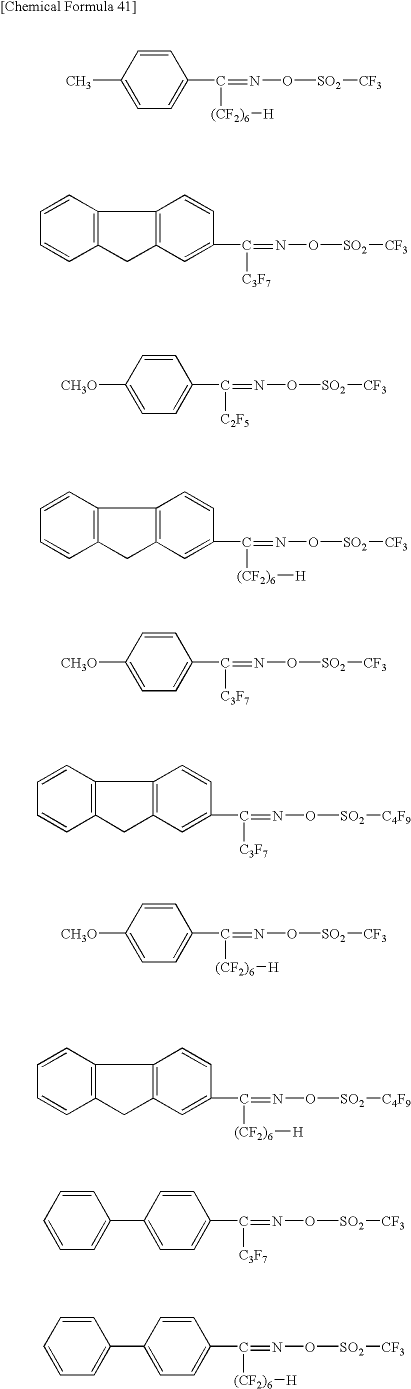

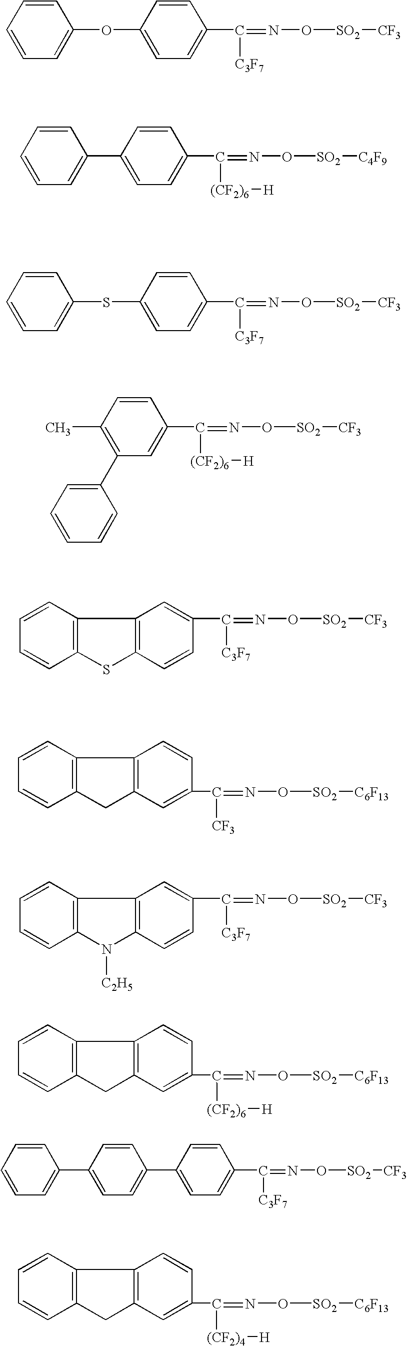

Also, particularly suitable examples of the oxime sulfonate-based acid generator include 4 compounds shown below.

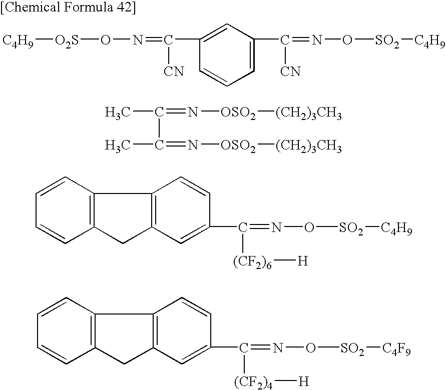

Among the diazomethane-based acid generators, specific examples of bisalkyl- or bisarylsulfonyldiazomethanes include bis(isopropylsulfonyl)diazomethane, bis(p-toluenesulfonyl)diazomethane, bis(1,1-dimethylethylsulfonyl)diazomethane, bis(cyclohexylsulfonyl)diazomethane, and bis(2,4-dimethylphenylsulfonyl)diazomethane.

Also, diazomethane-based acid generators disclosed in Japanese Unexamined Patent Application, First Publication No. Hei11-035551, Japanese Unexamined Patent Application, First Publication No. Hei11-035552, and Japanese Unexamined Patent Application, First Publication No. Hei11-035573 can be preferably used.

Examples of the poly(bis-sulfonyl)diazomethanes include 1,3 -bis(phenylsulfonyldiazomethylsulfonyl)propane, 1,4-bis(phenylsulfonyldiazomethylsulfonyl)butane, 1,6-bis(phenylsulfonyldiazomethylsulfonyl)hexane, 1,10-bis(phenylsulfonyldiazomethylsulfonyl)decane, 1,2-bis(cyclohexylsulfonyldiazomethylsulfonyl)ethane, 1,3-bis(cyclohexylsulfonyldiazomethylsulfonyl)propane, 1,6-bis(cyclohexylsulfonyldiazomethylsulfonyl)hexane, and 1,10-bis(cyclohexylsulfonyldiazomethylsulfonyl)decane, which are disclosed in Japanese Unexamined Patent Application, First Publication No. Hei11-322707.

As the component (B2), one kind selected from the above acid generators may be used alone, or two or more kinds may be used in combination.

The amount of the component (B) in the resist composition of the present invention is preferably within the range from 0.5 to 30 parts by mass, and more preferably from 1 to 20 parts by mass, relative to 100 parts by mass of the component (A). When the amount is within the range, a pattern can be sufficiently formed. Also, a uniform solution and excellent storage stability can be obtained. Therefore, an amount within the above range is preferable.

### <Component (D)>

In the resist composition of the present invention, for improving the resist pattern shape and the post exposure stability of the latent image formed by the pattern-wise exposure of the resist layer, it is preferable to add a nitrogen-containing organic compound (D) (hereinafter referred to as component (D)) as an optional component.

Since a multitude of these components (D) have already been proposed, any of these known compounds can be arbitrarily used. Of these, an aliphatic amine, particularly a secondary aliphatic amine or tertiary aliphatic amine is preferred. Here, the term “aliphatic” is a relative concept used in relation to the term “aromatic”, and defines a group or compound or the like that contains no aromaticity.

The term “aliphatic cyclic group (alicyclic group)” represents a monocyclic or polycyclic group that contains no aromaticity.

Examples of the aliphatic amine include an amine (alkylamine or alkylalcoholamine) wherein at least one of the hydrogen atoms of NH₃ is substituted with an alkyl or hydroxyalkyl group having 1 to 12 carbon atoms; and a cyclic amine.

Specific examples of the alkylamines or alkylalcoholamines include monoalkylamines such as n-hexylamine, n-heptylamine, n-octylamine, n-nonylamine, or n-decylamine; dialkylamines such as diethylamine, di-n-propylamine, di-n-heptylamine, di-n-octylamine, or dicyclohexylamine; trialkylamines such as trimethylamine, triethylamine, tri-n-propylamine, tri-n-butylamine, tri-n-hexylamine, tri-n-pentylamine, tri-n-heptylamine, tri-n-octylamine, tri-n-nonylamine, tri-n-decanylamine, or tri-n-dodecylamine; and alkylalcoholamines such as diethanolamine, triethanolamine, diisopropanolamine, triisopropanolamine, di-n-octanolamine, or tri-n-octanolamine. Among these amines, trialkylamines of 5 to 10 carbon atoms are preferable, tri-n-pentylamine and tri-n-octylamine are more preferable, and tri-n-pentylamine is most preferable.

Examples of the cyclic amine include a heterocyclic compound containing a nitrogen atom as a hetero atom. The heterocyclic compound may be a monocyclic compound (aliphatic monocyclic amine), or a polycyclic compound (aliphatic polycyclic amine).

Specific examples of the aliphatic monocyclic amines include piperidine, and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

These may be used either alone, or in combination of two or more different compounds.

The component (D) is typically used in a quantity within a range of 0.01 to 5.0 parts by weight, relative to 100 parts by weight of the component (A).

### <Optional Component>[Component (E)]

Furthermore, in the resist composition of the present invention, for preventing any deterioration in sensitivity, and improving the resist pattern shape and the post exposure stability of the latent image formed by the pattern-wise exposure of the resist layer, at least one compound (E) (hereinafter, referred to as component (E)) selected from the group consisting of an organic carboxylic acid, or a phosphorus oxo acid or derivative thereof can be added.

Suitable examples of organic carboxylic acids include acetic acid, malonic acid, citric acid, malic acid, succinic acid, benzoic acid, and salicylic acid.

Examples of phosphorus oxo acids include phosphoric acid, phosphonic acid and phosphinic acid. Among these, phosphonic acid is particularly preferable.

Examples of phosphorus oxo acid derivatives include esters in which a hydrogen atom within the above-mentioned oxo acids is substituted with a hydrocarbon group.

Examples of the hydrocarbon group include an alkyl group of 1 to 5 carbon atoms and an aryl group of 6 to 15 carbon atoms.

Examples of phosphoric acid derivatives include phosphate esters such as di-n-butyl phosphate and diphenyl phosphate.

Examples of phosphonic acid derivatives include phosphonate esters such as dimethyl phosphonate, di-n-butyl phosphonate, phenylphosphonic acid, diphenyl phosphonate and dibenzyl phosphonate.

Examples of phosphinic acid derivatives include phosphinic esters such as phenylphosphinic acid.

As the component (E), one type may be used alone, or two or more types may be used in combination.

As the component (E), an organic carboxylic acid is preferable, and salicylic acid is particularly preferable.

The component (E) is used in a quantity within a range of 0.01 to 5.0 parts by weight, relative to 100 parts by weight of the component (A).

### [Component (O)]

In the positive resist composition of the present invention, if desired, additives having miscibility, for example, additive resins for improving performance of a resist film, surfactants for improving coatability, dissolution inhibitors, plasticizers, stabilizers, colorants, antihalation agents, and dyes can be appropriately added.

### [Component (S)]

The resist composition according to the third aspect of the present invention can be prepared by dissolving materials in an organic solvent (hereinafter, sometimes referred to as component (S)).

The component (S) may be an organic solvent which can dissolve the respective components used in the present invention to give a uniform solution, and one or more kinds of organic solvents can be used, appropriately selected from those which have been conventionally known as a solvent for a chemically-amplified resist.

Examples thereof include lactones such as γ-butyrolactone; ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, dipropylene glycol; compounds having ester bonds such as ethylene glycol monoacetate, diethylene glycol monoacetate, propylene glycol monoacetate and dipropylene glycol monoacetate; and polyhydric alcohol derivatives including compounds having ether bonds such as monoalkyl ethers (for example, monomethyl ether, monoethyl ether, monopropyl ether and monobutyl ether) and monophenyl ether of the above polyhydric alcohols or the above compounds having ester bonds (of these, propylene glycol monomethyl ether acetate (PGMEA) and propylene glycol monomethyl ether (PGME) are preferable); cyclic ethers such as dioxane; esters such as methyl lactate, ethyl lactate (EL), methyl acetate, ethyl acetate, butyl acetate, methyl pyruvate, ethyl pyruvate, methyl methoxypropionate, ethyl ethoxypropionate; and aromatic organic solvents such as anisole, ethylbenzyl ether, cresylmethyl ether, diphenyl ether, dibenzyl ether, phenetole, butylphenyl ether, ethylbenzene, diethylbenzene, pentylbenzene, isopropylbenzene, toluene, xylene, cymene, and mesitylene.

These organic solvents may be used either alone, or as a mixed solvent of two or more different solvents.

Of these, propylene glycol monomethyl ether acetate (PGMEA), propylene glycol monomethyl ether (PGME), ethyl lactate (EL) and γ-butyrolactone are preferable.

Also, a mixed solvent obtained by mixing PGMEA and a polar solvent is preferable. The mixing ratio (mass ratio) of PGMEA to the polar solvent may be appropriately decided taking account of compatibility, and is preferably adjusted within a range of 1:9 to 9:1, and more preferably 2:8 to 8:2.

More specifically, in the case of using EL as the polar solvent, the mass ratio PGMEA:EL is preferably within a range of 1:9 to 9:1, and more preferably 2:8 to 8:2. Furthermore, in those cases of using PGME as the polar solvent, the mass ratio PGMEA:PGME is preferably within a range of 1:9 to 9:1, and more preferably 2:8 to 8:2.

Furthermore, as the component (S), mixed solvents of at least one of PGMEA and EL with γ-butyrolactone are also preferred. In such cases, the mass ratio of the former and latter components in the mixed solvents is preferably within a range of 70:30 to 95:5.

Furthermore, as the component (S), a mixed solvent of a mixture of PGMEA and PGME with γ-butyrolactone is also preferable.

There is no particular restriction on the quantity of the component (S), and the quantity should be set in accordance with the required coating film thickness within a concentration that enables favorable application of the solution to a substrate or the like. Typically, the quantity is set so that the solid fraction concentration within the resist composition falls within a range of 2 to 20% by weight, and still more preferably 5 to 15% by weight.

In a method of forming a resist pattern including immersion exposure, the resist composition of the present invention can be suitably used as a resist composition for immersion lithography, and can obtain excellent lithography properties. Also, in a method of forming a resist pattern including formation of a triple-layer resist laminate, the resist composition of the present invention can be suitably used as a positive resist composition for forming an upper-layer resist film, and can obtain excellent lithography properties.

The reasons include that the acid generator (B1) composed of the compound represented by the general formula (b1-2) used in the present invention effectively suppresses the absorption of light within a wavelength range of exposure (particularly a wavelength range of ArF excimer lasers), and has satisfactory solubility in an organic solvent (resist solvent) used for dissolving various resist components. Therefore, it is considered that the resist composition of the present invention can suppress the absorption of light due to including the component (B1), and transparency of the resist composition is enhanced.

Further, it is considered that the component (B1) has satisfactory dispersity in the resist film, and thus is distributed more uniformly in the resist film than conventional acid generators. Therefore, it is presumed that the acid generated from the component (B1) upon exposure can be dispersed more uniformly within the resist film as compared with conventional acid generators.

For the reasons described above, it is considered that, in the method of forming a resist pattern including immersion exposure, the resist composition of the present invention can be suitably used as a resist composition for immersion lithography, and can obtain excellent lithography properties. Also, it is considered that, in the method of forming a resist pattern including formation of a triple-layer resist laminate, the resist composition of the present invention can be suitably used as a positive resist composition for forming an upper-layer resist film, and can obtain excellent lithography properties.

According to the resist composition of the present invention, a resist pattern can be formed with excellence in terms of lithography properties such as line width roughness (LWR), the resist pattern shape (particularly surface roughness within the resist pattern), mask error factor (MEF), and exposure margin (EL margin).

Here, the MEF is a parameter that indicates how faithfully mask patterns of differing dimensions can be reproduced by using the same exposure dose with fixed pitch and changing the mask size (line width and space width). The closer the MEF value is to 1, the better the mask reproducibility.

The EL margin is a parameter that indicates the amount of change in the pattern size which is associated with the change in exposure dose. The larger the EL margin value, the smaller the amount of change.

### <<Method of Forming Resist Pattern>>

Next, the method of forming a resist pattern according to the fourth aspect of the present invention will be described below.

The method of forming a resist pattern according to the present invention includes: forming a resist film on a substrate by using a resist composition described above in the third aspect of the present invention; exposing the resist film; and alkali-developing the resist film to form a resist pattern.

As examples of the method of forming a resist pattern of the present invention, there are a method of forming a resist pattern including the step of conducting immersion exposure, and a method of forming a resist pattern including a step of forming a triple-layer resist laminate. These will be explained below.

### <Method of Forming Resist Pattern Including Immersion Exposure>

First, the resist composition (in this case, hereinafter sometimes referred to as resist composition for immersion lithography) of the third aspect of the present invention is applied on a substrate such as a silicon wafer using a spinner, and then prebake (post apply bake (PAB) treatment) is conducted, thereby forming a resist film.

Here, an organic or inorganic antireflective film may be provided between the substrate and the applied layer of the resist composition, and a two-layer laminate thus obtained may also be used.

Further, a top coat can be provided on the resist film.

There is no particular restriction on the top coat, and those which are conventionally used for immersion lithography can be used. For example, protective films disclosed in International Publication WO 2005/019937 and International Publication WO 2004/074937; and protective films formed from a composition in which a main-chain cyclic resin containing a group represented by -Q-NH—SO₂—R⁵ (wherein, Q represents a linear or branched alkylene group of 1 to 5 carbon atoms; and R⁵ represents a fluorinated alkyl group), and/or —CO—O—R⁷ (wherein, R⁷ represents a fluorinated alkyl group) is dissolved in an organic solvent (an alcohol-based solvent such as isobutanol) can be used.

Here, the expression “main-chain cyclic resin” means a resin containing a structural unit (hereinafter, referred to as “main-chain cyclic structural unit”) which contains a monocyclic or polycyclic ring structure, wherein at least one, preferably two or more carbon atoms on the ring of the ring structure constitutes a main chain.

As the main-chain cyclic structural unit, structural units derived from polycycloolefins (polycyclic olefins) and dicarboxylic acid anhydride-containing structural units can be used. Of these, it is preferable to include a structural unit derived from a polycycloolefin (polycylic olefin, and preferably norbornene or the like) in the main chain.

It is preferable that the top coat provided on the resist film be soluble in an alkali developing solution.

The steps so far can be conducted by using a conventional method. It is preferable that the operating condition or the like be arbitrarily set according to the formulation and properties of the resist composition for immersion lithography used.

In the method of forming a resist pattern including immersion exposure, subsequently, a resist film obtained above is selectively exposed by immersion exposure (liquid immersion lithography) through a desirable mask pattern. Here, the region between the resist film obtained and the lens at the lowermost point of the exposure apparatus is pre-filled with a solvent (immersion solvent) that has a larger refractive index than the refractive index of air, and then, keeping such a condition, the exposure (immersion exposure) is conducted.

There is no particular restriction on the wavelength used for the exposure, and the exposure can be conducted using radiation such as ArF excimer lasers, KrF excimer lasers, and F₂ lasers. The resist composition for immersion lithography of the present invention is effective for a KrF excimer laser or an ArF excimer laser, and particularly effective for an ArF excimer laser.

As described above, in the method of forming a resist pattern of the present invention, the region between the resist film (or the top coat) and the lens at the lowermost point of the exposure apparatus is filled with an immersion solvent at the time of exposure, and the exposure (immersion exposure) is conducted while keeping such a condition.

Here, the immersion solvent is preferably a solvent that has a refractive index larger than the refractive index of air but smaller than the refractive index of the resist film formed from the resist composition for immersion lithography. There is no restriction on the refractive index of the immersion solvent, as long as the solvent has a refractive index within the above range.

Examples of the solvent which has a refractive index larger than that of air but smaller than that of a resist film include water, fluorine-based inactive liquid, and a silicon-based solvent.

Specific examples of the fluorine-based inactive liquid include a liquid which has a fluorine-based compound as a main component, such as C₃HC1₂F₅, C₄F₉OCH₃, C₄F₉OC₂H₅, and C₅H₃F₇. The fluorine-based inactive liquid preferably has a boiling point within a range of 70 to 180° C., and more preferably 80 to 160° C. If the fluorine-based inactive liquid has a boiling point within the above range, the solvent used for the immersion lithography can be removed by a convenient method after exposure, and consequently it is preferable.

The fluorine-based inactive liquid is particularly preferably a perfluoroalkyl compound in which all hydrogen atoms of the alkyl groups are substituted with fluorine atoms. Examples of the perfluoroalkyl compounds include perfluoroalkylether compounds and perfluoroalkylamine compounds.

Specific examples of the perfluoroalkylether compounds include a perfluoro(2-butyl-tetrahydrofuran) (boiling point: 102° C.), and specific examples of the perfluoroalkylamine compounds include a perfluorotributylamine (boiling point: 174° C.).

Since the resist composition of the present invention is particularly resistant to any adverse effects caused by water, and thus excels in sensitivity and shape of the resist pattern profile, water is preferably used as the immersion solvent. Furthermore, water is also preferred in terms of cost, safety, environmental friendliness, and versatility.

After conducting immersion exposure, post exposure bake (PEB) treatment is conducted, followed by a developing treatment with an alkali developing solution composed of an aqueous alkali solution. Then, water rinse is preferably conducted with pure water. This water rinse can be conducted, for example, by dripping or spraying water onto the surface of the substrate while rotating the substrate, and washes away the developing solution and those portions of the resist composition for immersion lithography that have been dissolved by the developing solution. Further, drying treatment is conducted, thereby obtaining a resist pattern in which the resist film (coated film of the resist composition for immersion lithography) is patterned with the shape according to the mask pattern.

### <Method of Forming Resist Pattern Including Formation of Triple-Layer Resist Laminate>

The method of forming a resist pattern including formation of a triple-layer resist laminate includes steps of: forming a triple-layer resist laminate which includes forming a lower-layer organic film which can be dry-etched on a substrate, applying and then heating a material which includes an Si atom on the lower-layer organic film to form an interlayer, and forming an upper-layer resist film on the interlayer using the resist composition of the third aspect of the present invention, thereby forming a triple-layer resist laminate; and forming a resist pattern which includes exposing the upper-layer resist film, and developing the upper-layer resist film, thereby forming a resist pattern.

### Examples

The following is a description of examples of the present invention, although the scope of the present invention is by no way limited by these examples.

### Example 1

### Synthesis of Compound (b1-21)

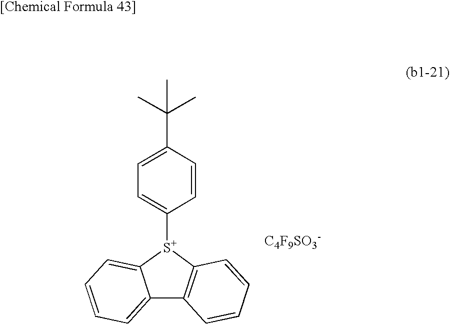

15.00 g of bis(4-tert-butylphenyl)iodonium perfluorobutanesulfonate, 4.38 g of dibenzothiophene, and 0.33 g of copper benzoanate (II) were dissolved in 30 g of chlorobenzene, and reacted for 2 hours at 110° C. After the reaction, the resulting solution was concentrated and dried, and then dissolved in 35 g of dichloromethane. The dichloromethane solution was washed using water, and then 130 g of hexane was added thereto as a poor solvent, thereby obtaining a crystal. The crystal thus obtained was dried under reduced pressure at room temperature, thereby obtaining 8.0 g of the intended compound.

The compound was analyzed by using ¹H-NMR and ¹⁹F-NMR.

¹H-NMR (CDCl₃, 400 MHz, internal standard: tetramethylsilane): δ(ppm)=8.19 (s, 4H, Hd+Hg), 7.87 (t, 2H, Hf), 7.66 (t, 2H, He), 7.62 (d, 2H, Hb), 7.54 (d, 2H, Hc), 1.28 (s, 9H, Ha).

¹⁹F-NMR (CDCl₃, 376 MHz): δ(ppm)=80.9, 114.5, 121.4, 125.8.

From the results described above, it could be confirmed that the compound had the structure shown below.

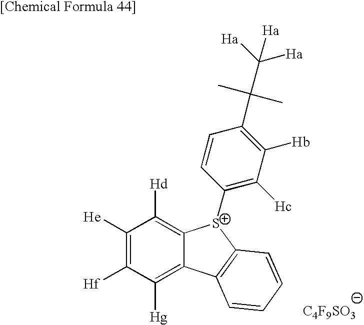

### Example 2

### Synthesis of Compound (b1-24)

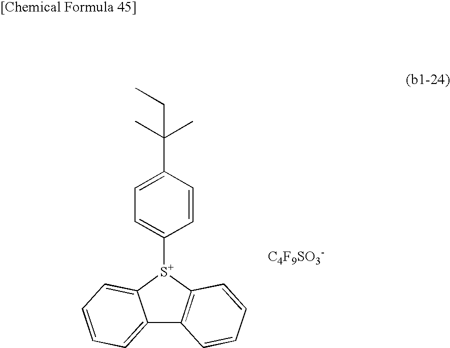

10.00 g of bis(4-tert-pentylphenyl)iodonium perfluorobutanesulfonate, 2.81 g of dibenzothiophene, and 0.11 g of copper benzoanate (II) were dissolved in 15 g of chlorobenzene, and reacted for 1 hour at 110° C. After the reaction, hexane was added thereto as a poor solvent, thereby obtaining a crystal. The crystal thus obtained was dried under reduced pressure at room temperature, thereby obtaining 3.21 g of the intended compound.

The compound was analyzed by using ¹H-NMR and ¹⁹F-NMR.

¹H-NMR (CDCl₃, 400 MHz, internal standard: tetramethylsilane): δ(ppm)=8.24 (d, 2H, Hf), 8.11(d, 2H, Hi), 7.84(t, 2H, Hh), 7.61(t, 2H, Hg), 7.58(d, 2H, Hd), 7.46(d, 2H, He), 1.60(q, 2H, Hb), 1.23(s, 6H, Hc), 0.63(t, 3H, Ha).

¹⁹F-NMR (CDCl₃, 376 MHz): δ(ppm)=75.9, 110.1, 116.8, 121.1.

From the results described above, it could be confirmed that the compound had the structure shown below.

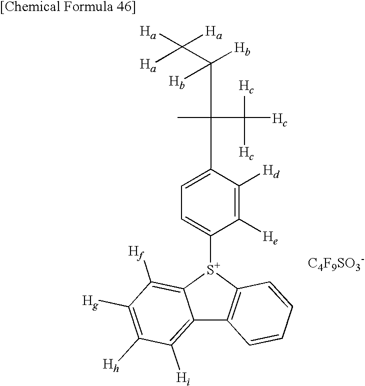

### <Evaluation of Solubility>

With respect to the aforementioned compounds (b1-21) and (b1-24), and di(1-naphthyl)phenylsulfonium nonafluorobutanesulfonate ((B)-2), the solubility was evaluated in the following manner.

The solution of propylene glycol monomethyl ether acetate (PGMEA), propylene glycol monomethyl ether (PGME), or ethyl lactate (EL) with each compound described above was prepared, while changing the concentration. After the preparation, each solution was stirred, and then the concentration in which each acid generator was completely dissolved was measured.

The results are shown in Table 1. It could be confirmed that the aforementioned compounds (b1-21) and (b1-24) according to the first aspect of the present invention have excellent solubility in PGMEA, PGME, and EL, which were commonly-used resist solvents, when compared with di(1-naphthyl)phenylsulfonium nonafluorobutanesulfonate ((B)-2).

TABLE 1

| | Example 1 | Example 2 | |
|---|---|---|---|
| | (b1-21) | (b1-24) | (B)-2 |
| Solubility in PGMEA (% by weight) | 5 | 15 | 2 |
| Solubility in PGME (% by weight) | >20 | >20 | 5 |
| Solubility in EL (% by weight) | 7 | >20 | 5 |

### Examples 3 and 4, Comparative Examples 1and 2

### Resin Component (A)

The polymer (A)-1 of the component (A) used in Examples 3 and 4, and Comparative Examples 1 and 2 is shown below.

Here, the weight average molecular weight (Mw) and the dispersity (Mw/Mn) of the polymer (A)-1 are also described below. The weight average molecular weight (Mw) and the dispersity (Mw/Mn) were determined by the polystyrene equivalent molecular weight measured by gel permeation chromatography (GPC).

Also, the compositional ratio was determined by carbon NMR. In the chemical formula shown below, the value on the bottom-right of each structural unit means the proportion (mol %) of each structural unit in the polymer.

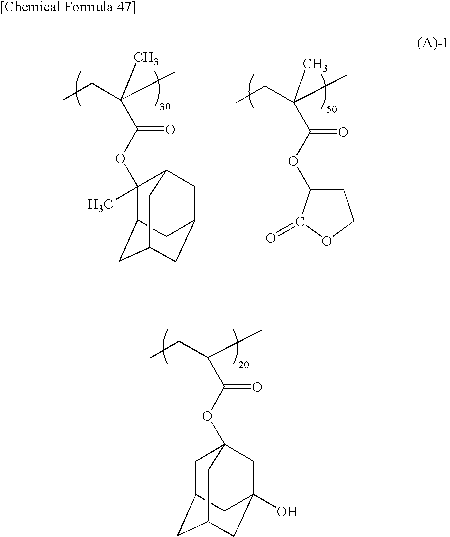

(Mw: 10,000, Mw/Mn: 2.0; the polymer (A)-1 was synthesized using monomers from which each structural units were derived, by a conventional dropwise polymerization method.)

### <Preparation of Positive Resist Composition Solution>

The components shown in Table 2 were mixed and dissolved, thereby providing a positive resist composition solution. Here, the term “−” in the Table 2 means that nothing is blended.

TABLE 2

| | (A) | (B) | | (D) | (E) | (S) | |
|---|---|---|---|---|---|---|---|
| Example 3 | (A)-1 | (B)-1 | | (D)-1 | (E)-1 | (S)-1 | (S)-2 |
| | [100] | [12.0] | | [0.54] | [1.32] | [10] | [2200] |
| Example 4 | (A)-1 | (B)-5 | | (D)-1 | (E)-1 | (S)-1 | (S)-2 |
| | [100] | [12.5] | | [0.54] | [1.32] | [10] | [2200] |
| Com- | (A)-1 | (B)-2 | | (D)-1 | (E)-1 | (S)-1 | (S)-2 |
| parative | [100] | [13.0] | | [0.54] | [1.32] | [10] | [2200] |
| Example 1 | | | | | | | |
| Com- | (A)-1 | (B)-3 | (B)-4 | (D)-2 | — | — | (S)-2 |
| parative | [100] | [3.50] | [1.00] | [0.30] | | | [2200] |
| Example 2 | | | | | | | |

In Table 2, the abbreviations mean the following. Also, the values within the brackets [ ] mean the blending amount (parts by weight).

(B)-1: the acid generator represented by the chemical formula (b-21) shown below (the compound of Example 1)

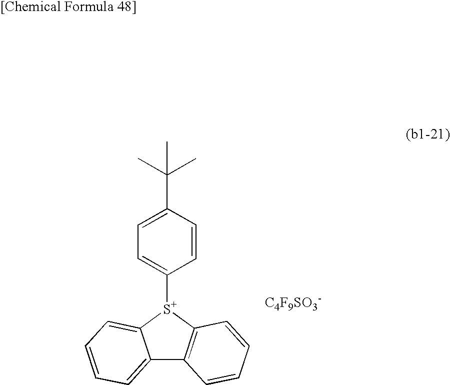

(B)-2: di(1-naphthyl)phenylsulfonium nonafluorobutanesulfonate

(B)-3: triphenylsulfonium nonafluorobuthanesulfonate

(B)-4: (4-methylphenyl)diphenylsulfonium trifluoromethanesulfonate

(B)-5: the acid generator represented by the chemical formula (b1-24) shown below (the compound of Example 2)

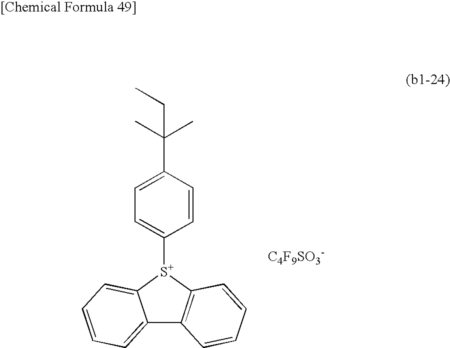

(D)-1: tri-n-pentylamine

(D)-2: triethanolamine

(E)-1: salicylic acid

(S)-1: γ-butyrolactone

(S)-2: a mixture solvent of PGMEA/PGME=6/4 (mass ratio).

### <Evaluation of Lithography Properties>

Resist patterns were formed using the positive resist composition solutions thus obtained, and the following lithography properties were evaluated.

### [Formation of Resist Pattern]

An organic anti-reflection film composition (product name: ARC29A, manufactured by Brewer Science Ltd.) was applied onto an 8-inch silicon wafer using a spinner, and the composition was then baked at 205° C. for 60 seconds and dried, thereby forming an organic anti-reflection film having a film thickness of 77 nm. Then, the positive resist composition solution obtained above was applied onto the anti-reflection film using a spinner, and then a prebake (PAB) treatment was conducted on a hotplate at a temperature shown in Table 3 for 60 seconds and dried, thereby forming a resist film having a film thickness of 150 nm.

Subsequently, the obtained resist film was selectively exposed by an ArF excimer laser (193 nm), using an ArF exposure apparatus “NSR-S302” (manufactured by Nikon; numerical aperture (NA)=0.60, ⅔ annual illumination) through a mask pattern. Thereafter, a post exposure bake (PEB) treatment was conducted at a temperature shown in Table 3 for 60 seconds, followed by development for 30 seconds at 23° C. in a 2.38% by weight aqueous solution of tetramethylammonium hydroxide (TMAH). Then, the resist was washed for 30 seconds with pure water, and dried by shaking, thereby forming a resist pattern (L/S pattern) with a line and space (1:1). The optimum exposure dose (sensitivity: Eop, mJ/cm²) for forming the L/S pattern with a line width of 120 nm and a pitch of 240 nm was determined.

### [Line Width Roughness (LWR)]

With respect to each of L/S patterns with a line width of 120 nm and a pitch of 240 nm which was formed using the aforementioned Eop, the line width was measured at 5 points in a longitudinal direction of the line using a length measuring SEM (product name: “S-9220”; manufactured by Hitachi, Ltd.). From the results, 3-fold value (3s) of standard deviation (s) was calculated as an indicator which indicates LWR. The results are shown in Table 3.

Here, smaller value of 3s means that a resist pattern having smaller roughness of the line width and more uniform width could be obtained.

TABLE 3

| | | | | Comparative | Comparative |
|---|---|---|---|---|---|
| | Example 3 | | Example 4 | Example 1 | Example 2 |
| PAB temperature | 110 | 115 | 110 | 110 | 115 |
| (° C.) | | | | | |
| PEB temperature | 110 | 115 | 110 | 110 | 115 |
| (° C.) | | | | | |
| Eop (mJ/cm²) | 84.0 | 66.0 | 70.0 | 39.8 | 18.6 |
| LWR (nm) | 7.8 | 8.1 | 7.3 | 9.1 | 11.0 |

From the results shown in Table 3, it could be confirmed that a resist pattern with a smaller value of LWR and more uniform width could be obtained in Examples 3 and 4 of the present invention, when compared with Comparative Examples 1 and 2.

Further, it could be confirmed that, with respect to the exposure margin (EL margin), each of the resist compositions of Examples 3 and 4 of the present invention was at the same level as those of Comparative Examples 1 and 2.

From the results described above, it could be confirmed that the resist compositions of Examples 3 and 4 of the present invention could obtain excellent lithography properties.

### Example 5 and Comparative Example 3

Example 5 is an example of the case in which the method of forming a resist pattern including formation of a triple-layer resist laminate was conducted using the compound of Example 1 of the present invention as the acid generator of the positive resist composition for forming an upper-layer resist film.

Comparative Example 3 is a comparative example in which the positive resist composition solution used in Comparative Example 1 was used in the method of forming a resist pattern including formation of a triple-layer resist laminate.

### [Formation of Resist Pattern]

A resist laminate was produced by the following procedure, and a resist pattern was formed using the resist laminate.

Firstly, an undercoating material (product name: BLC750, manufactured by Tokyo Ohka Kogyo Co., Ltd.) was applied onto an 8-inch silicon wafer using a spinner, and then soft-baked for 90 seconds at 230° C., thereby forming a lower-layer organic film with a film thickness of 270 nm.

Subsequently, a composition for forming a hard mask (composition composed of a copolymer of phenyl silsesquioxane, hydrogen silsesquioxane, methyl silsesquioxane, and methyl propionat-1-yl silsesquioxane dissolved in a mixed solvent of PGMEA and EL (mass ratio 6:4), (solid fraction concentration: 2.5% by weight)) was applied on the lower layer using a spinner, soft-baked for 90 seconds at 90° C., and then baked for 90 seconds at 250° C., thereby forming a hard mask layer (interlayer) with a film thickness of 30 nm.

Thereafter, the same positive resist composition solution as that of Example 3 or Comparative Example 1 was applied on the interlayer using a spinner, conducting a prebake (PAB) treatment on a hotplate for 60 seconds at each temperature shown in Table 4, and then dried, thereby forming a positive resist layer (upper-layer resist film) with a film thickness of 150 nm. Accordingly, a resist laminate in which a resist layer with a triple-layer structure was laminated on a substrate was obtained.

Subsequently, the obtained resist laminate was selectively exposed by an ArF excimer laser (193 nm), using an ArF exposure apparatus “NSR-S302” (manufactured by Nikon; numerical aperture (NA)=0.60, ⅔ annual illumination) through a mask pattern. Thereafter, a post exposure bake (PEB) treatment was conducted at a temperature shown in Table 4 for 60 seconds, followed by development for 30 seconds at 23° C. in a 2.38% by weight aqueous solution of tetramethylammonium hydroxide (TMAH). Then, the resist was washed for 30 seconds with pure water, and dried by shaking, thereby forming a resist pattern (L/S pattern) with a line and space (1:1). The optimum exposure dose (sensitivity: Eop, mJ/cm²) for forming the L/S pattern with a line width of 120 nm and a pitch of 240 nm was determined.

### [Line Width Roughness (LWR)]

In the same manner as Example 3, the value of 3s was calculated an indicator which indicates LWR. The results are shown in Table 4.

TABLE 4

| | | Comparative |
|---|---|---|
| | Example 5 | Example 3 |
| PAB temperature (° C.) | 110 | 110 |
| PEB temperature (° C.) | 110 | 110 |
| Eop (mJ/cm²) | 76.2 | 37.0 |
| LWR (nm) | 8.7 | 9.2 |

From the results shown in Table 4, it could be confirmed that, even in the case of the laminated resist, a resist pattern with a smaller value of LWR and more uniform width could be obtained in Example 5 of the present invention, when compared with Comparative Example 3.

From the results described above, it could be confirmed that, in the method of forming a resist pattern including formation of a triple-layer resist laminate, the resist composition of Example 5 of the present invention had excellent compatibility with the interlayer, and hence, footing of the resist pattern could be suppressed.

### INDUSTRIAL APPLICABILITY

According to the present invention, there can be provided a novel compound suitable as an acid generator for a resist composition, an acid generator composed of the compound, a resist composition including the acid generator, and a method of forming a resist pattern using the resist composition. Therefore, the present invention is extremely useful industrially.

1. A compound represented by a general formula (b1-2) shown below: 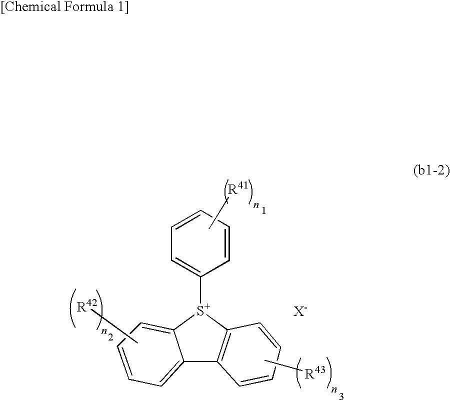
(wherein, R⁴¹, R⁴², and R⁴³ each independently represents an alkyl group, an acetyl group, an alkoxy group, a carboxyl group, or a hydroxyalkyl group; n₁ represents an integer of 0 to 3; n₂ and n₃ each independently represents an integer of 0 to 3, where not all of n₁, n₂, and n₃ are simultaneously 0; and X⁻ represents an anion).

2. The compound according to claim 1, wherein said X⁻ represents a halogenated alkylsulfonate ion.

3. An acid generator composed of the compound described in claim 1 or 2.

4. A resist composition, comprising a base component (A) which exhibits changed solubility in an alkali developing solution under action of an acid, and an acid generator component (B) which generates an acid upon exposure, wherein
the acid generator component (B) comprises an acid generator (B1) composed of a compound represented by a general formula (b1-2) shown below: 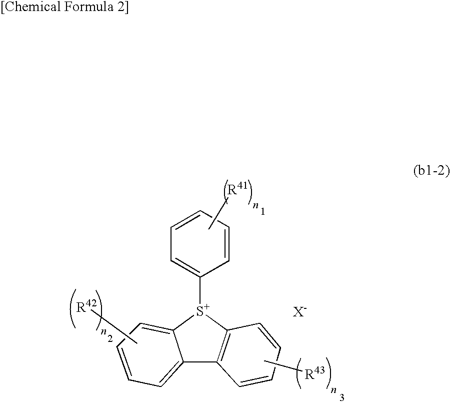
(wherein, R⁴¹, R⁴², and R⁴³ each independently represents an alkyl group, an acetyl group, an alkoxy group, a carboxyl group, or a hydroxyalkyl group; n₁ represents an integer of 0 to 3; n₂ and n₃ each independently represents an integer of 0 to 3, where not all of n₁, n₂, and n₃ are simultaneously 0; and X⁻ represents an anion).

5. The compound according to claim 4, wherein said X⁻ represents a halogenated alkylsulfonate ion.

6. The resist composition according to claim 4, wherein the content of the acid generator (B1) is within the range of 1 to 30 parts by weight, relative to 100 parts by weight of the base component (A).

7. The resist composition according to claim 4, wherein the base component (A) is a base component which exhibits increased solubility in an alkali developing solution under action of acid.

8. The resist composition according to claim 7, wherein the base component (A) is a resin component (A1) which exhibits increased solubility in an alkali developing solution under action of acid.

9. The resist composition according to claim 8, wherein the resin component (A1) comprises a structural unit (a1) derived from an acrylate ester having an acid dissociable, dissolution inhibiting group.

10. The resist composition according to claim 9, wherein the resin component (A1) further comprises a structural unit (a2) derived from an acrylate ester having a lactone-containing cyclic group.

11. The resist composition according to claim 9, wherein the resin component (A1) further comprises a structural unit (a3) derived from an acrylate ester having a polar group-containing aliphatic hydrocarbon group.

12. The resist composition according to claim 10, wherein the resin component (A1) further comprises a structural unit (a3) derived from an acrylate ester having a polar group-containing aliphatic hydrocarbon group.

13. The resist composition according to claim 4, further comprising a nitrogen-containing organic compound (D).

14. A method of forming a resist pattern, comprising: forming a resist film on a substrate using a resist composition of any one of claims 4 to 13; exposing the resist film; and developing the resist film to form a resist pattern.
